(19) United States
(12) Patent Application Publication
(10) Pub. No.: US 2026/0191971 A1
(43) Pub. Date: Jul. 9, 2026

(54) CHARGE-ALTERING NUCLEIC ACID TRANSPORTERS WITH BETA-AMIDO CARBONATE BACKBONES

(71) Applicant: The Board of Trustees of the Leland Stanford Junior University, Stanford, CA (US)

(72) Inventors: Zhijian Li, Stanford, CA (US); Robert M. Waymouth, Stanford, CA (US); Paul Wender, Stanford, CA (US); Howard Chang, Stanford, CA (US); Laura Amaya, Stanford, CA (US)

(73) Assignee: The Board of Trustees of the Leland Stanford Junior University, Stanford, CA (US)

(21) Appl. No.: 19/128,412

(22) PCT Filed: Nov. 8, 2023
(86) PCT No.: PCT/US23/79051

§ 371 Date: May 8, 2025

Related U.S. Application Data
(60) Provisional application No. 63/423,541, filed on Nov. 8, 2022.

(51) Int. Cl. A61K 47/59; A61K 9/00; A61K 47/54; C12N 15/88

(52) U.S. Cl. CPC A61K 47/595; A61K 9/0019; A61K 47/549; C12N 15/88

(57) ABSTRACT

Provided are polymeric compounds of Formula I which include cationic poly(alpha-amino ester) monomers and lipid functionalized beta-amido carbonate (bAC) monomers, and related compositions and methods for transport and delivery of polyanions, including nucleic acids, into cells in vitro, ex vivo, and in vivo.

This application claims priority to U.S. Provisional application Ser. No. 63/423,541 filed Nov. 8, 2022, the content of which is hereby incorporated by reference in its entirety.

### BACKGROUND

Efficient delivery vehicles for transporting nucleic acids to target cells and tissues are needed to realize the full potential of nucleic acid-based technologies, including for example RNA-based vaccines and immunotherapies such as chimeric antigen receptor T cell therapy (“CAR-T therapy”) and in vivo T cell reprogramming.

Polymeric systems represent a class of nucleic acid delivery system. The present invention addresses the need for improved polymeric nucleic acid delivery vehicles.

### BRIEF SUMMARY

The present invention provides polymeric delivery vehicles for transport and delivery into cells of polyanions, including nucleic acids. Thus, in aspects, the invention provides polymeric nucleic acid transporters. The delivery vehicles described here are copolymers which include repeating units of cationic poly(alpha-amino ester) monomers and lipid funcctionalized carbonate monomers having a beta-amido carbonate (bAC) backbone, including copolymers in which one or both monomer units are statistically mixed.

In aspects, the invention provides a compound of Formula I that includes one or two bAC units, LP1, LP2, a poly(alpha-amino ester) unit, IM, and an initiator or ligand moiety, A, covalently attached to one of the bAC units, optionally via a linker moiety L1.

Accordingly, provided are compounds of Formula I

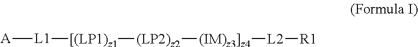

where A is a substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl; or A is a ligand moiety that binds to and is internalized by a cell surface receptor, optionally a saccharide, a disaccharide, an oligosaccharide, a liposaccharide, a lipid, a peptide, an antibody, or a small molecule;
R1 is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
L1 and L2 are optional and each is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP1 and LP2 are each independently a lipid functionalized beta-amido carbonate (bAC) monomer having a structure defined by:

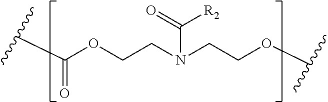

where R2 is branched or unbranched, substituted or unsubstituted C₅-C₃₀ alkyl or heteroalkyl, which may be fully saturated, mono- or polyunsaturated;
IM is an alpha aminoester monomer unit having a structure defined by

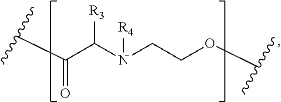

where R3 is dihydrogen or methyl and R4 is dihydrogen or substituted or unsubstituted C₂-C₅ heteroalkyl, wherein the heteroatom is nitrogen and wherein the substituent, if present, is carbonyl;
and
z1 and z2 are independently 0 to 100, optionally from 5-20, and at least one of z1 or z2 is not 0, z3 is 2-100, optionally 5-20, and z4 is 1-100.

In aspects, IM is

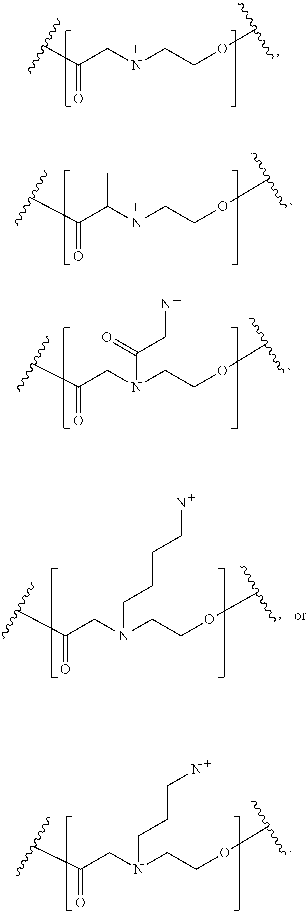

In each of the formulas above, N+ is intended to signify +NH2.

The compound may also include where A is alkoxyaryl, optionally methoxyphenyl or ethoxyphenyl, and L1 is a bond. In aspects, A is substituted or unsubstituted phenyl.

The compound may also include where A is a ligand moiety that binds to a cell surface receptor selected from a saccharide, a disaccharide, an oligosaccharide, a liposaccharide, a lipid, a peptide, an antibody, or a small molecule, optionally fingolimod or a fingolimod analog.

The compound may also include where A is glucose (beta-D-glucopyranoside) or galactose (alpha-D-galactopyranose).

In aspects, A is

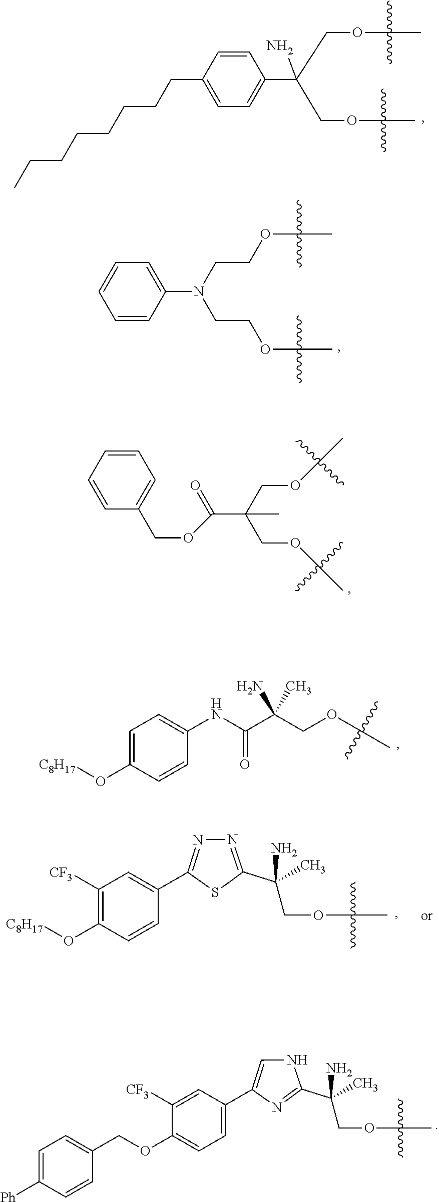

The compound may also include where each R2 is independently a branched or unbranched C₈-C₃₀ alkyl, which may be fully saturated, mono- or polyunsaturated.

The compound may also include where each R2 is independently an isoprenoid lipid or a sterol such as cholesterol and its derivatives.

The compound may also include where each R2 is independently stearyl, oleyl, linoleyl, dodecyl, nonenyl, isoprenoid, or cholesterol.

The compound may also include where each R2 is independently unbranched C₈-C₃₀ alkyl, which may be fully saturated, mono- or polyunsaturated, or a branched C₈-C₃₀ alkyl, which may be fully saturated, mono- or polyunsaturated.

In aspects, R² is

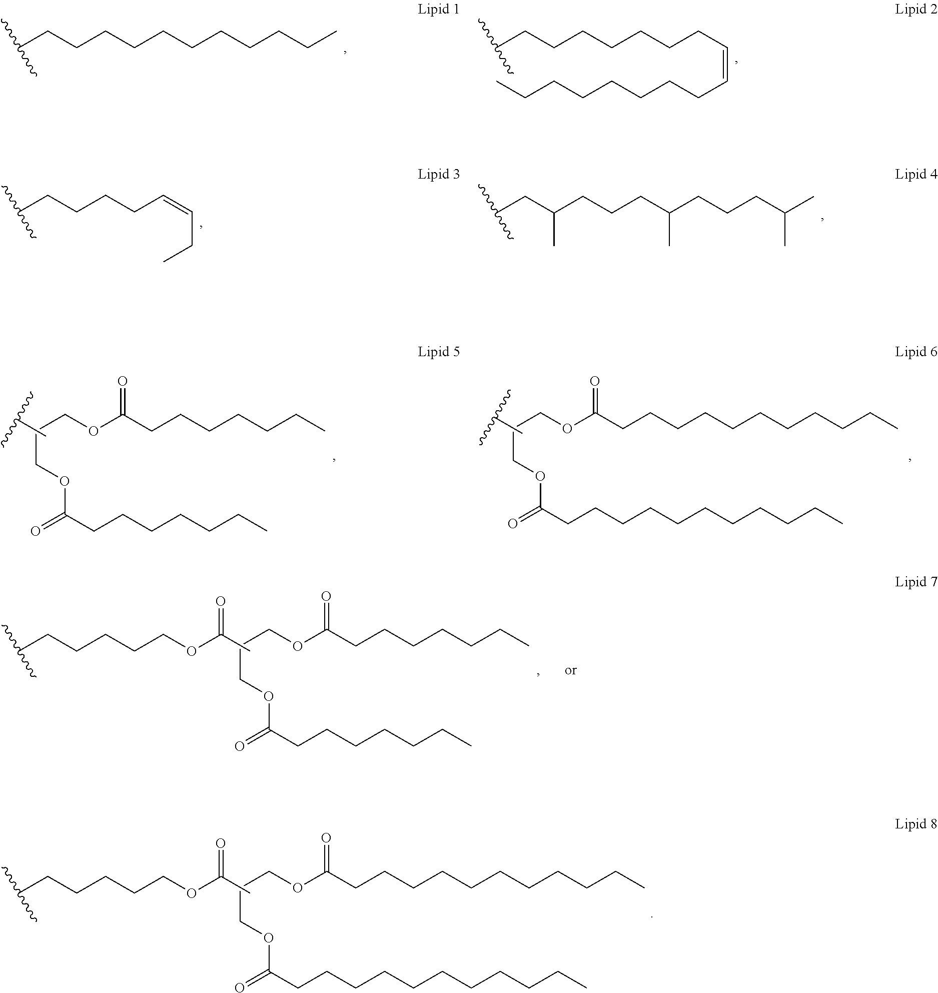

In aspects, R² is isoamyl, phytyl, or farnesyl.

In aspects, the compound has the structure of Formula Ia:

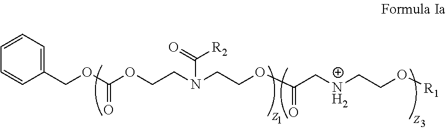

wherein R1 is hydrogen and R2 is branched or unbranched, substituted or unsubstituted C₈-C₃₀ alkyl or heteroalkyl, which may be fully saturated, mono- or polyunsaturated.

In aspects, a compound of Formula Ia may also include where R2 is unsubstituted branched or unbranched C₈-C₃₀ alkyl, which may be fully saturated, mono- or polyunsaturated.

In aspects, a compound of Formula Ia may also include where R2 is substituted or unsubstituted branched or unbranched C₈-C₃₀ heteroalkyl and the heteroatom is oxygen (O).

In aspects, a compound of Formula Ia may also include where R2 is substituted branched or unbranched C₈-C₃₀ alkyl or heteroalkyl containing from 1 to 6 substituents, optionally selected from one or more of methyl and acyl.

In aspects, a compound of Formula Ia may also include where R2 is

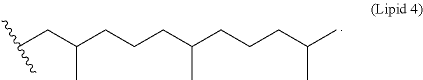 [SMILES: *CC(C)CCCC(C)CCCC(C)C]

In aspects, the compound may also include where R2 is Lipid 4, z1 is 5-25 and z3 is 10-20; optionally where z1 is 17 and z3 is 10, which compound is designated bAC-4a, or where z1 is 10 and z3 is 11, which compound is designated bAC-4b; or where z1 is 9 and z3 is 16, which compound is designated bAC-4c.

In aspects, a compound of Formula Ia may also include where R2 is

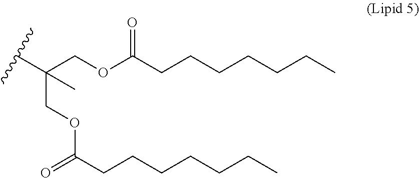 [SMILES: *C(C)(COC(=O)CCCCCCC)COC(=O)CCCCCCC]

In aspects, the compound may also include where z1 is 5-25 and z3 is 10-20; optionally where z1 is 19 and z3 is 9, which compound is designated bAC-5a; or where z1 is 10 and z3 is 9, which compound is designated bAC-5b; or where z1 is 10 and z3 is 14, which compound is designated bAC-5c.

In aspects, a compound of Formula Ia may also include where R2 is

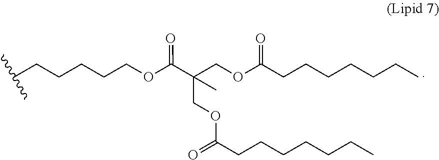 [SMILES: *CCCCCOC(=O)C(C)(COC(=O)CCCCCCC)COC(=O)CCCCCCC]

In aspects, the compound may also include where z1 is 5-25 and z3 is 10-20; optionally where z1 is 17 and z3 is 10, which compound is designated bAC-7a; or where z1 is 10 and z3 is 10, which compound is designated bAC-7b; or where z1 is 12 and z3 is 18, which compound is designated bAC-7c.

In aspects, a compound of Formula Ia may also include where R2 is

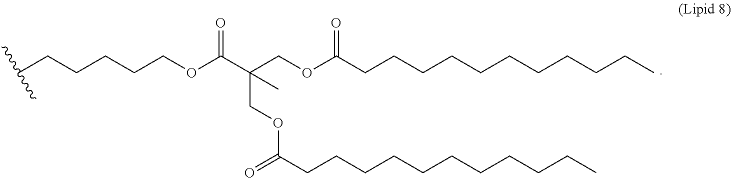 [SMILES: *CCCCCOC(=O)C(C)(COC(=O)CCCCCCCCCCC)COC(=O)CCCCCCCCCCC]

In aspects, the compound may also include where z1 is 5-25 and z3 is 5-20; optionally where z1 is 16 and z3 is 9, which compound is designated bAC-8a; or where z1 is 10 and z3 is 9, which compound is designated bAC-8b; or where z1 is 8 and z3 is 18, which compound is designated bAC-8c.

The invention also provides compositions comprising a compound of Formula I, or subformula thereof, non-covalently attached to a nucleic acid. In aspects, the composition comprises nanoparticulate particles of a compound of Formula I, or subformula thereof, non-covalently attached to a nucleic acid. In aspects, the composition may also include where the charge ratio (N:P) of the complexes or nanoparticulate particles in the composition is about 10:1. In aspects, the composition may also include where the nucleic acid is selected from the group consisting of RNA or DNA. In aspects, the composition may also include where the nucleic acid is selected from the group consisting of messenger RNA (mRNA), small interference RNA (siRNA), short hairpin RNA (shRNA), micro RNA (miRNA), guide RNA (gRNA), CRISPR RNA (crRNA), transactivating RNA (tracrRNA), circular RNA (circRNA), self-amplifying RNA, plasmid DNA (pDNA), minicircle DNA, and genomic DNA (gNDA), and combinations of two or more of any of the foregoing. In aspects, the composition may also include where the nucleic acid is messenger RNA (mRNA).

The invention also provides pharmaceutical compositions comprising a compound of Formula I, or subformula thereof, non-covalently attached to a nucleic acid, and a pharmaceutically acceptable carrier or excipient. In aspects, the pharmaceutical composition may also include where the composition is a vaccine composition, optionally, where the carrier or excipient includes an immunological adjuvant. In aspects, the pharmaceutical composition may also include where the mRNA encodes a chimeric antigen receptor (CAR).

The invention also provides methods for transfecting a nucleic acid into a cell, where the method includes contacting the cell with a complex comprising a compound of Formula I, or subformula thereof, non-covalently attached to a nucleic acid. In aspects, the method includes where the contacting is in vivo, in vitro, or ex vivo. In aspects, the method may also include where the cell is a eukaryotic cell, a mammalian cell, a tumor cell, or a lymphoid cell. In aspects, the method may also include where the cell is a B or T lymphocyte, a dendritic cell, or a macrophage cell. In aspects, the method may also include where the cell is a B lymphocyte, a dendritic cell, a macrophage cell, or a T lymphocyte, optionally where the T lymphocyte is a CD8+ T cell or a CD4+ T cell.

The invention also provides methods for transfecting a nucleic acid into a splenocyte cell of a mammal, the method includes administering to the mammal a composition or pharmaceutical composition comprising a compound of Formula I, or subformula thereof, non-covalently attached to a nucleic acid, optionally where the nucleic acid is an mRNA. In aspects, the mRNA encodes a chimeric antigen receptor (CAR).

The invention also provides methods for treating an autoimmune disease or disorder, a cancer, or an infectious disease, the method including administering the pharmaceutical composition as described herein to a subject in need of therapy for an autoimmune disease or disorder, a cancer, or an infectious disease, optionally where administration is by a parenteral route, further optionally where administration is by an intravenous route, optionally where the subject is human.

The invention also provides methods for preventing a disease or disorder, the method including administering the pharmaceutical composition as described herein to a subject in need of therapy for the disease or disorder, where the nucleic acid encodes one or more antigenic or immunogenic epitopes or peptides, optionally where administration is by a parenteral route, further optionally where administration is by an intravenous route, optionally where the subject is human. The method may also include where the disease or disorder is an infectious disease. The method may also include where the infectious disease is caused by an infectious agent, optionally where the infectious agent is a virus, a bacterium, or a protozoa.

The invention also provides for the use of the pharmaceutical compositions described herein, which may include their use in a method for treating an autoimmune disease or disorder, a cancer, or an infectious disease in a subject, optionally where the subject is human. Also provided is the use of the pharmaceutical compositions described herein in a method for preventing an infectious disease in a subject, optionally wherein the subject is human.

The invention also provides for the use of a composition or pharmaceutical composition comprising a compound of Formula I, or subformula thereof, non-covalently attached to a nucleic acid, for transfecting a nucleic acid into a splenocyte cell of a mammal, optionally where the nucleic acid is an mRNA. In aspects, the mRNA encodes a chimeric antigen receptor (CAR).

Other technical features may be readily apparent to one skilled in the art from the following figures, descriptions, and claims.

### BRIEF DESCRIPTION OF THE DRAWINGS

FIG. 1A illustrates an example of a generic CART structure with MTC backbone used as a comparator in experiments described herein.

FIG. 1B illustrates the beta-amido carbonate backbone of a compound of Formula Ia.

FIG. 2A Schematic representation of monomer synthesis. (D) Chemical structures of bAC library. (E) MTC analogs library. (F) Heatmap of particle size (top) and backbone comparison (bottom) (G) Heatmap of eGFP expression comparing (top) and eGFP+ and AUC levels among best CARTs (bottom). (n=4, bars represent SEM).

FIG. 2B illustrates a synthetic scheme for a representative compound of the present invention, bAC-1a.

FIG. 2C is a bar graph showing Jurkat transfection of EGFP mRNA using as the delivery vehicle either bAC-1a, its MTC analog, MTC-1A, or a reference CART, ONA. (n=4, bars represent SEM) ****P<0.0001. Structures of MTC-1A and ONA are shown above the bar graph.

FIG. 2D illustrates the monomer structures used to prepare representative bAC compounds of Formula Ia. In the figure, “m” and “n” correspond to z1 and z3 of Formula Ia.

FIG. 2E illustrates the structures of the MTC analogs used for comparative analyses with the respective bAC molecules.

FIG. 2F is a bar graph showing particle sizes of selected bAC:RNA complexes compared to their MTC analogs. (n=4, bars represent SEM) *P<0.05, **P<0.01, ***P<0.001, ****P<0.0001. Differences between groups were considered significant for P values <0.05. NS, not significant.

FIG. 2G is a bar graph showing EGFP expression for selected bAC molecules compared to a reference (ONA), determined as either percentage EGFP+ cells or AUC. Statistical significance was calculated using one-way ANOVA followed by Tukey's test. (n=4, bars represent SEM) *P<0.05, **P<0.01, ***P<0.001, ****P<0.0001. Differences between groups were considered significant for P values <0.05. NS, not significant.

FIG. 3A is a set of two bar graphs showing bAC compound screening in primary human T cells, percentage of EGFP (left) and AUC fluorescent signal (right) 24 h after transfection.

FIG. 3B is a set of two bar graphs showing percentage of EGFP expressing cells (top) and AUC fluorescent signal (bottom) 24 h after transfection of EGFP mRNA using the bAC compound indicated across the top of the bar graphs, and compared to each of the respective MTC analogs. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 3C is a bar graph showing percentage of EGFP expressing cells following transfection of EGFP mRNA with bAC-7c (right bar in each pair) or ONA (left bar in each pair) in resting T cells, or T cells transfected at 6, 24, or 48 hours after activation. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 3D is a bar graph showing percentage of EGFP expressing cells following transfection of EGFP mRNA using the indicated bAC compound, bAC-4, bAC-5, bAC-7, or bAC-8, each having three different monomer lengths, a, b, or c. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 3E is a bar graph showing percentage of EGFP expressing cells following transfection of EGFP mRNA with bAC-7c (right 4 bars) or ONA (left 4 bars) using 50, 100, 200, or 400 ng mRNA. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 3F is a bar graph showing percentage of EGFP expressing cells following transfection of EGFP mRNA with bAC-7b or using electroporation (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 4A is a line graph illustrating the correlation between mRNA uptake measured as percentage of cy5 positive cells and protein translation measured as percentage of EGFP positive cells.

FIG. 4B is a bar graph showing percentage of cy5 positive cells as an indicator of mRNA uptake following transfection with the indicated bAC compound, bAC-1b, bAC-4b, bAC-7b, or bAC-8b, and their respective MTC analogs. Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 5A is a schematic representation of mRNA delivery in vivo to assess biodistribution. 5 ug of luciferase mRNA is complexed with the bAC compound and delivered retro-orbitally. Bioluminescence imaging is acquired 6 hours after transfection.

FIG. 5B is a bar graph showing bioluminescence after delivery of luciferase mRNA complexed with bAC-4 or bAC-7 of the indicated block lengths, a-c. Picture inset shows a representative image of whole body image analysis. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 5C is a bar graph showing the increase in transfection efficiency for bAC compounds, bAC-4b and bAC-7b, compared to their respective MTC analogs. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 5D illustrates the biodistribution of representative bAC compounds, bAC-4b and bAC-7c, compared to a reference MTC compound, ONA.

FIG. 5E is a bar graph with photo insert of the luminescence signal in the spleens of mice administered representative bAC compounds, bAC-4b and bAC-7c, compared to a reference MTC compound, ONA. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 5F is a schematic representation of mRNA delivery in vivo to assess cell-specificity. 15 ug of Cre mRNA were complexed with each CART and delivered retro-orbitally, flow cytometric analysis of isolated spleens was performed 48 hours after transfection.

FIG. 5G is a bar graph showing Cre-mediated recombination in district cellular subsets. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 5H illustrates the percentage of Cre-mediated recombination in CD4+ cells in vivo. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 5I illustrates the percentage of Cre-mediated recombination in CD8+ cells in vivo. (n=4, bars represent SEM). Statistical significance was calculated using one-way ANOVA followed by Tukey's test. *P<0.05, **P<0.01, ***P<0.001, ****P<0.0001. Differences between groups were considered significant for P values <0.05. NS, not significant.

FIG. 6A is a schematic representation of the assay used to assess generation of cytotoxic CAR-T cells.

FIG. 6B is a bar graph representing the percentage of anti-hCD19 expression 20 hours post-transfection of anti-hCD19 mRNA with ONA and bAC-7c (n=6, bars represent SD). Statistical significance was calculated using Mann Whitney test.

FIG. 6C shows the percentage of cells expressing the degranulation marker CD107a, and each of the activation markers, IFN-γ and TNF-α, after co-culture with Nalm6-GL cells at 1:4 effector:target (E:T) ratio. Data in the groups treated with bAC/mRNA or ONA/mRNA complexes was normalized by the baseline marker expressions of untransfected T cells in the same co-culture well.

FIG. 6D shows the percentage of cell killing (Nalm6-GL cells) after co-culture with anti-hCD19 expressing CAR T cells at 10:1 effector:target (E:T) ratio. Statistical significance was calculated using Two-way ANOVA in d and e. (n=6, bars represent median).

FIG. 7A is a schematic representation of mRNA delivery in vivo to assess T cell specific luciferase expression.

FIG. 7B is a bar graph showing quantitation of luminescence signal in untreated splenic T cells or 6 hours after transfection of 5 ug mRNA luciferase with ONA or bAC-7c (n=3, bars represent SD). Statistical significance was calculated using unpaired t-test with Welch's correction.

FIG. 7C is a bar graph showing the effect of mRNA dose in isolated splenic T cells 6 hours after transfection with bAC-7c (n=3, bars represent SD). Statistical significance was calculated using unpaired t-test with Welch's correction.

FIG. 8A is a bar graph showing results of preclinical toxicology blood analysis (blood metabolites AST, ALT, Alkaline Phosphatase) 24 hours after delivery of 7.5 mg of luciferase mRNA formulated with bAC-7c (shaded bars) or MC3 LNP (white bars).

FIG. 8B is a bar graph showing results of preclinical toxicology blood analysis (blood metabolites ABUN, Calcium, Phosphorus) 24 hours after delivery of 7.5 mg of luciferase mRNA formulated with bAC-7c (shaded bars) or MC3 LNP (white bars).

FIG. 8C is a bar graph showing results of preclinical toxicology blood analysis (blood counts) 24 hours after delivery of 7.5 mg of luciferase mRNA formulated with bAC-7c (shaded bars) or MC3 LNP (white bars).

FIG. 8D is a bar graph showing inflammatory cytokines 24 hours before (white bars) and after (shaded bars) delivery of 7.5 mg of luciferase mRNA formulated with bAC-7c.

### DETAILED DESCRIPTION

The disclosure provides polymeric delivery vehicles for transport and delivery into cells of polyanions, including nucleic acids. In aspects, provided are copolymer-based nucleic acid delivery vehicles. The polymeric delivery vehicles are derived from lipid-functionalized “bAC” carbonate monomers and poly(alpha-amino ester) monomers. Also provided are related compositions and methods. The polymer compounds described here advantageously provide efficient nucleic acid delivery to target cells, as exemplified in a particularly difficult cell model system, that of T lymphocytes, which is presented as a proof of concept of the general utility of the compounds described here as nucleic acid transporters. As described in more detail below, the disclosure provides compound of Formula I, and subformulas thereof, characterized by high efficiency in vitro and in vivo expression of mRNA delivered to T lymphocytes, including both a T cell line and primary T cells isolated from human blood. The poly(alpha-amino ester) component degrades through a unique pH-dependent intramolecular rearrangement to generate nontoxic degradation products. The carbonate segment of the poly(alpha-amino ester) component degrades through hydrolysis and decarboxylation, with non-toxic byproducts. Accordingly, the compounds are both biocompatible and biodegradable, as demonstrated by pre-clinical toxicity assays, making them ideal for clinical translation.

The nucleic acid transporters described here are copolymers, also referred to interchangeably herein as “co-oligomers” or “compounds”, which include repeating units of cationic poly(alpha-amino ester) monomers and lipid functionalized monomers having a beta-amido carbonate (bAC) backbone. The corresponding units of the copolymers derived from these monomers are referred to as “constitutional units” or “monomer units” or simply “units”.

The copolymers described here may be synthesized in a block architecture, where the units are distributed as blocks of identical monomer units, rather than statistically, or they may be may be synthesized in a statistical architecture. The nomenclature poly(A-stat-B) or polyA-block-polyB is adopted herein to differentiate statistical versus block copolymers, in accordance with Hodge et al., Pure Appl. Chem. 2020 92(5):797-813, where such differentiation is warranted. It is understood that where a formula does not specify either a stat or block architecture, both are envisioned as being encompassed by the formula. For example, compounds of Formula I as described herein may be polymers of either block or statistical architecture.

The polymer formulas described here are presented as graphical representations of an “ideal” form of the polymer. For example, while the graphical representations may present the repeating monomer units as connected to each other in the same orientation, e.g., “head-to-tail”, it is understood that in the actual polymer there may also be some “head-to-head” and/or “tail-to-tail” dyads.

Provided are compounds (also referred to herein as “copolymers”) of Formula I:

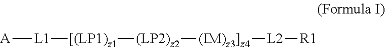

wherein
A is a substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl; or A is a ligand moiety that binds to and is internalized by a cell surface receptor, optionally a saccharide, a disaccharide, an oligosaccharide, a liposaccharide, a lipid, a peptide, an antibody, or a small molecule;
R1 is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
L1 and L2 are optional and each is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP1 and LP2 are each independently a functionalized beta-amido carbonate (bAC) monomer unit having a structure defined by

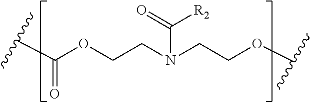

where R2 is branched or unbranched, substituted or unsubstituted C₈-C₃₀ alkyl or heteroalkyl, which may be fully saturated, mono- or polyunsaturated;
IM is an alpha aminoester (AAE) monomer unit having a structure defined by

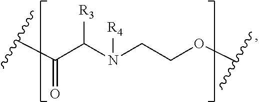

where R3 is dihydrogen or methyl and R4 is dihydrogen or substituted or unsubstituted C₂-C₅ heteroalkyl, wherein the heteroatom is nitrogen and wherein the substituent, if present, is carbonyl;
and
z1 and z2 are independently 0 to 100, optionally from 5-20, and at least one of z1 or z2 is not 0, z3 is 2-100, optionally 5-20, and z4 is 1-100.

In aspects, A is a terminal initiator moiety or ligand moiety covalently attached to a terminal lipophilic polymer block via an optional alkylene linker moiety, as defined herein. Preferably, the ligand moiety binds to a cell surface receptor, and may comprise, for example, a saccharide, a disaccharide, an oligosaccharide, a liposaccharide, a lipid, a peptide, an antibody, or a small molecule. As used in this context, the term “small molecule” refers to organic molecules of low molecular weight, on the order of less than about 1000 daltons, and as distinguished from “large molecules” which generally cannot rapidly diffuse across cell membranes, including, for example, nucleic acids, proteins, and polysaccharides.

In aspects, A is phenyl and L1 is C₁-C₃ alkoxy, optionally methoxy or ethoxy.

In aspects, A is alkoxyaryl and L1 is a bond, optionally methoxyphenyl or ethoxyphenyl.

In aspects, L1 and L2 are each independently a bond or an alkylene linker, optionally comprising one or more functional groups, as defined herein.

In aspects, IM is

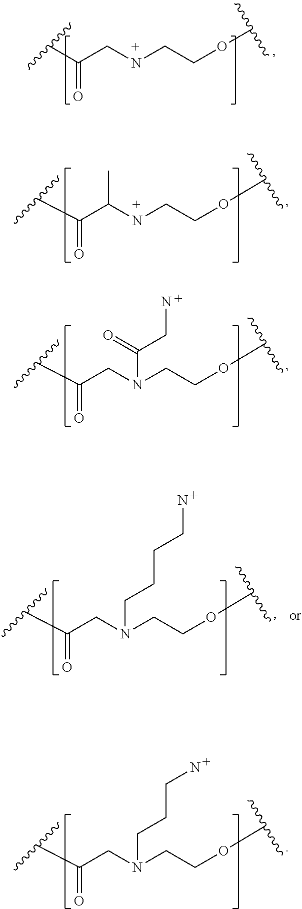

In some aspects, R₂ is branched substituted C₈-C₃₀ alkyl. The term “branched” in this context is intended to include a “forked” structure, as shown infra, e.g., Lipid 7 or Lipid 8. In some aspects, R2 is an isoprenoid lipid or a sterol such as cholesterol or a derivative thereof. In aspects, R2 is stearyl, oleyl, linoleyl, dodecyl, nonenyl, or cholesterol. In aspects where R2 is a substituted C₈-C₃₀ alkyl or heteroalkyl, R2 may include a diester or triester moiety. In aspects, R₂ is butyloctanyl, cholesteryl, dodecyl, linolenyl, linoleyl, nonenyl, oleyl, stearyl, or tocopheryl.

In aspects, R² is

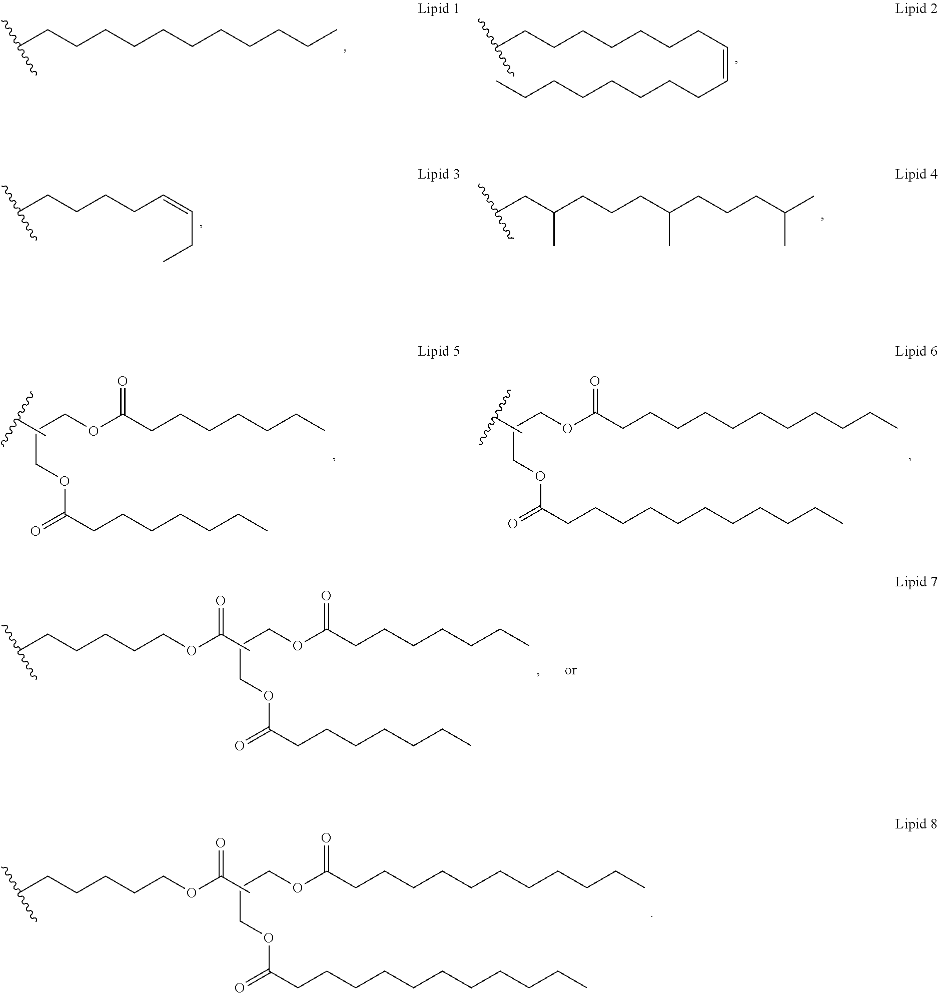

In aspects, provided is a compound of Formula Ia:

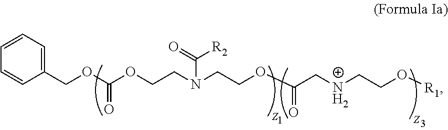

wherein R1 is hydrogen and R2 is a branched or unbranched, substituted or unsubstituted C₈-C₃₀ alkyl or heteroalkyl, which may be fully saturated, mono- or polyunsaturated. In aspects, the heteroatom, if present is O, and if substituted, the branched or unbranched C₈-C₃₀ alkyl or heteroalkyl contains from 1 to 6 substituents selected from one or more of methyl and acyl.

In aspects, provided is a compound of Formula Ia wherein R2 is Lipid 1, Lipid 2, Lipid 3, Lipid 4, Lipid 5, Lipid 6, Lipid 7, or Lipid 8, as defined above.

In aspects, provided is a compound of Formula Ia wherein R1 is H and R2 is Lipid 1, Lipid 2, Lipid 3, Lipid 4, Lipid 5, Lipid 6, Lipid 7, or Lipid 8, as defined above.

In aspects, provided is a compound of Formula Ia wherein R2 is Lipid 1, Lipid 2, Lipid 3, Lipid 4, Lipid 5, Lipid 6, Lipid 7, or Lipid 8, as defined above, and wherein z1 and z3 are each independently 5-100, 5-50, 5-25, 5-20, or 5-15.

In aspects, provided is a compound of Formula Ia wherein R1 is H and R2 is Lipid 1, Lipid 2, Lipid 3, Lipid 4, Lipid 5, Lipid 6, Lipid 7, or Lipid 8, as defined above, and wherein z1 and z3 are each independently 5-100, 5-50, 5-25, 5-20, or 5-15.

TABLE 1
Exemplary Compounds of Formula Ia

| Compound | R₁ | R₂ | z₁ | z₃ |
|---|---|---|---|---|
| bAC-1a | H | 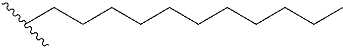 [SMILES: *CCCCCCCCCCC] | 17 | 10 |
| bAC-1b | H | 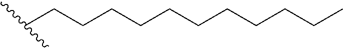 [SMILES: *CCCCCCCCCCC] | 10 | 10 |
| bAC-1c | H | 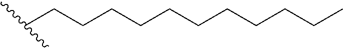 [SMILES: *CCCCCCCCCCC] | 10 | 16 |
| bAC-2a | H | 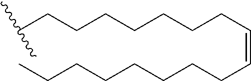 [SMILES: *CCCCCCC/C=C\CCCCCCCC] | 16 | 8 |
| bAC-2b | H | 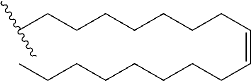 [SMILES: *CCCCCCC/C=C\CCCCCCCC] | 10 | 10 |
| bAC-2c | H | 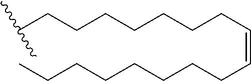 [SMILES: *CCCCCCC/C=C\CCCCCCCC] | 10 | 17 |
| bAC-3a | H | 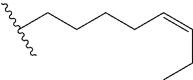 [SMILES: *CCCC/C=C\CC] | 17 | 10 |
| bAC-3b | H | 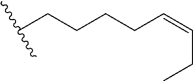 [SMILES: *CCCC/C=C\CC] | 11 | 10 |
| bAC-3c | H | 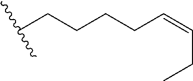 [SMILES: *CCCC/C=C\CC] | 11 | 17 |
| bAC-4a | H | 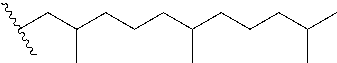 [SMILES: *CC(C)CCCC(C)CCCC(C)C] | 17 | 10 |
| bAC-4b | H | 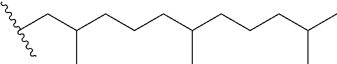 [SMILES: *CC(C)CCCC(C)CCCC(C)C] | 10 | 11 |
| bAC-4c | H | 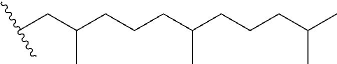 [SMILES: *CC(C)CCCC(C)CCCC(C)C] | 9 | 16 |
| bAC-5a | H | 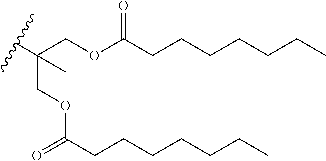 [SMILES: *C(C)(COC(=O)CCCCCCC)COC(=O)CCCCCCC] | 19 | 9 |
| bAC-5b | H | 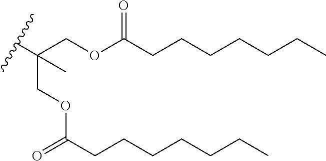 [SMILES: *C(C)(COC(=O)CCCCCCC)COC(=O)CCCCCCC] | 10 | 9 |
| bAC-5c | H | 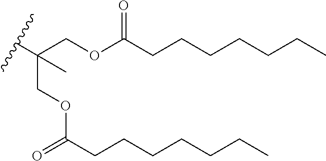 [SMILES: *C(C)(COC(=O)CCCCCCC)COC(=O)CCCCCCC] | 10 | 14 |
| bAC-6a | H | 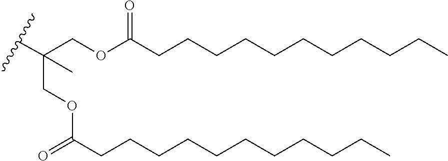 [SMILES: *C(C)(COC(=O)CCCCCCCCCCC)COC(=O)CCCCCCCCCCC] | 19 | 10 |
| bAC-6b | H | 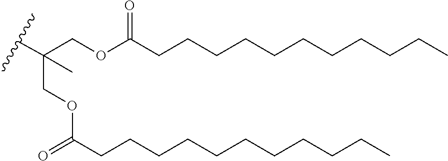 [SMILES: *C(C)(COC(=O)CCCCCCCCCCC)COC(=O)CCCCCCCCCCC] | 10 | 9 |
| bAC-6c | H | 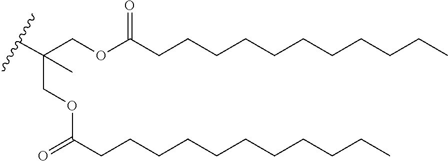 [SMILES: *C(C)(COC(=O)CCCCCCCCCCC)COC(=O)CCCCCCCCCCC] | 8 | 18 |
| bAC-7a | H | 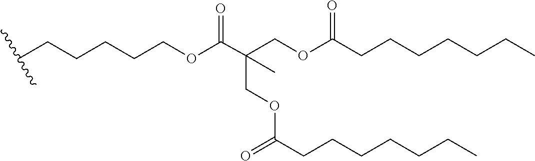 [SMILES: *CCCCCOC(=O)C(C)(COC(=O)CCCCCCC)COC(=O)CCCCCCC] | 17 | 10 |
| bAC-7b | H | 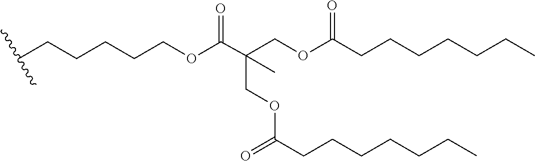 [SMILES: *CCCCCOC(=O)C(C)(COC(=O)CCCCCCC)COC(=O)CCCCCCC] | 10 | 10 |
| bAC-7c | H | 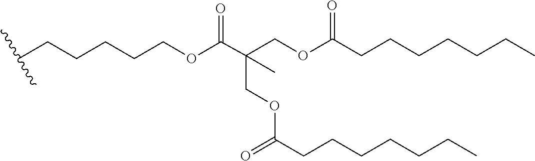 [SMILES: *CCCCCOC(=O)C(C)(COC(=O)CCCCCCC)COC(=O)CCCCCCC] | 12 | 18 |
| bAC-8a | H | 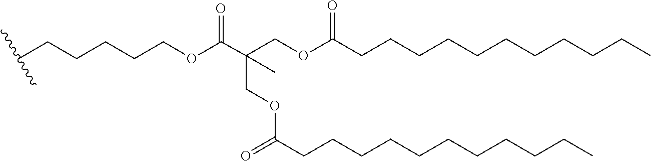 [SMILES: *CCCCCOC(=O)C(C)(COC(=O)CCCCCCCCCCC)COC(=O)CCCCCCCCCCC] | 16 | 9 |
| bAC-8b | H | 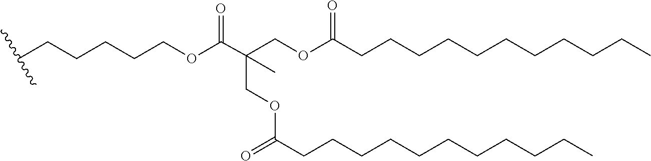 [SMILES: *CCCCCOC(=O)C(C)(COC(=O)CCCCCCCCCCC)COC(=O)CCCCCCCCCCC] | 10 | 9 |
| bAC-8c | H | 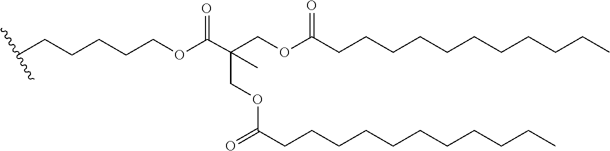 [SMILES: *CCCCCOC(=O)C(C)(COC(=O)CCCCCCCCCCC)COC(=O)CCCCCCCCCCC] | 8 | 18 |

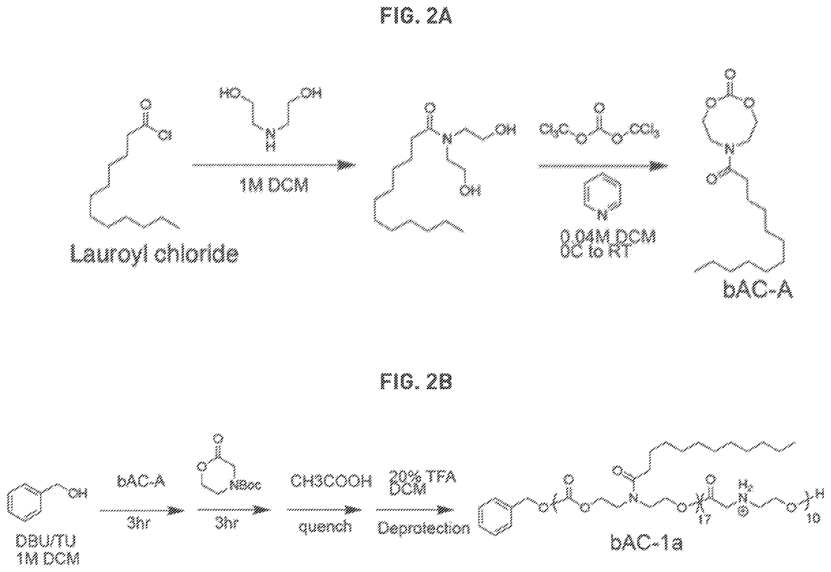

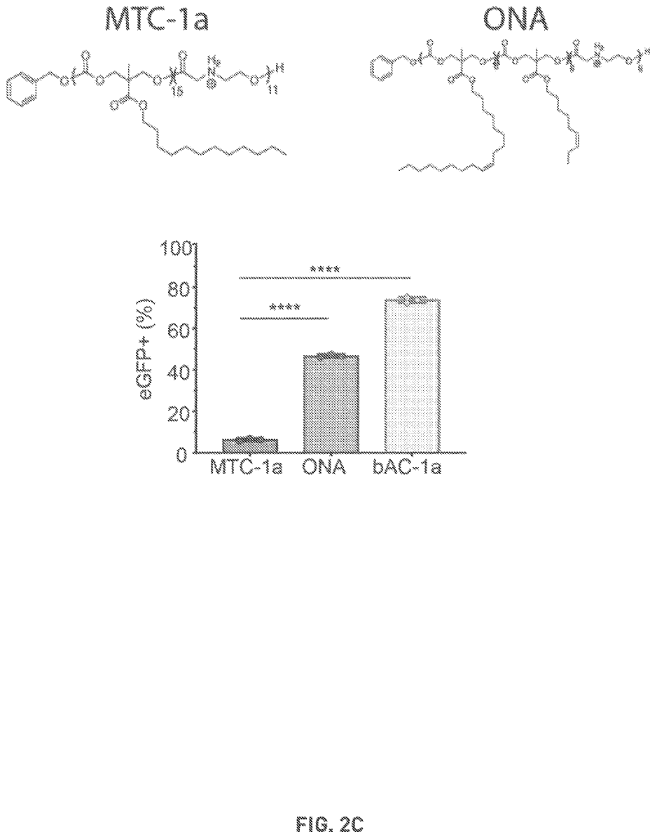

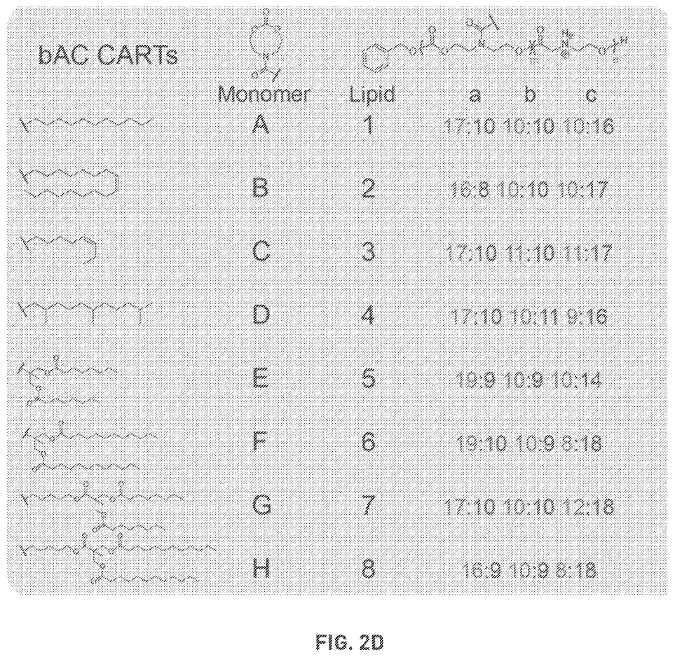

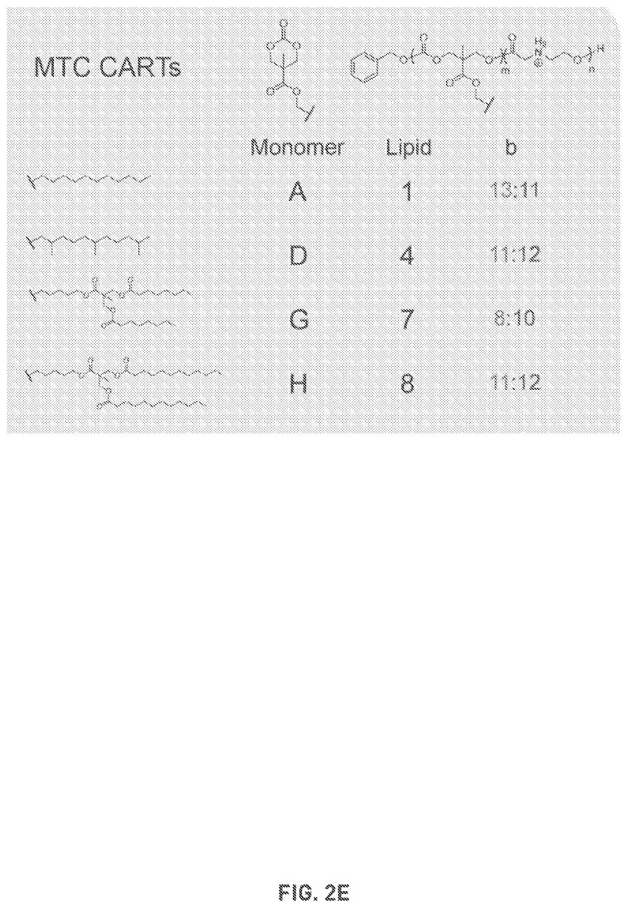

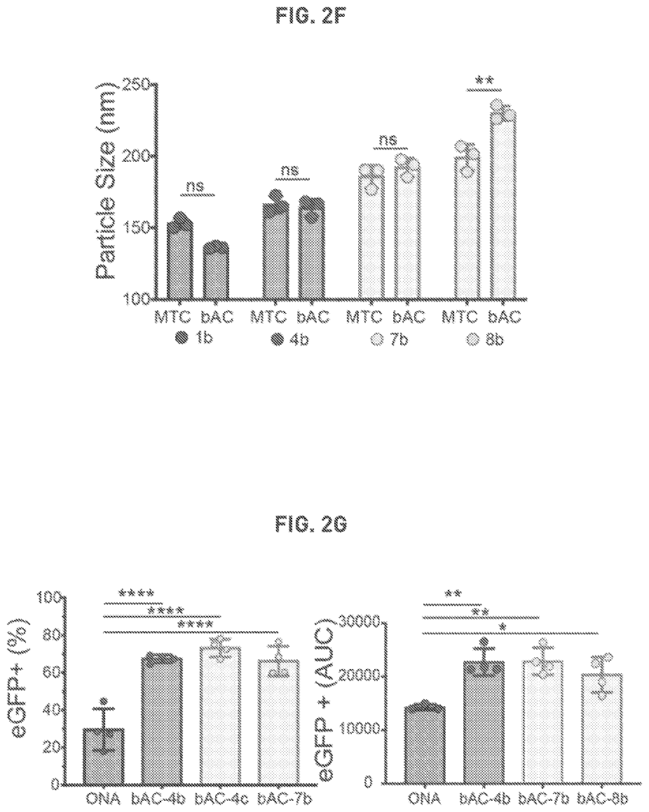

A synthetic scheme for a representative compound of Formula Ia is shown schematically in FIG. 2A and FIG. 2B. Briefly, the cyclic ester monomer was functionalized with a lauryl lipid, to form a bAC monomer, referred to in the figure as “bAC-A”. As shown in the figure, the functionalized diol was obtained in quantitative yield by coupling lauroyl chloride with diethanolamine. By screening various cyclization reagents and conditions, yields of 45-50% were obtained using a slow triphosgene addition in diluted diol reactants. Next, as illustrated in FIG. 2B, bAC-A was copolymerized with Boc protected morpholinone blocks, with benzyl alcohol as the initiator using the organocatalytic ring opening polymerization (OROP) methodology as previously described in Blake et al., J. Am Chem Soc. 2014 136:9252-9255 and Blake et al., Chem Sci 2020 11:2951. Following TFA deprotection, the resulting bAC compound (also referred to here in as “bAC CARTs”), designated “bAC-1a” in the figure, contained about 17 dodecyl lipid units and 10 cationic poly(alpha-amino ester) units.

A detailed synthetic method for a specific compound of Formula I designated “bAC-1a” is as follows. Compounds were prepared out of glove box using standard Schlenk link techniques. The procedure to synthesize Boc-protected bAC-1a starts by adding bAC-A monomer (49.27 mg, 17 equivalent, 157 umol) and thiourea catalyst (7 mg) into a glass GPC vial with a stir bar. The vial was flushed with nitrogen, and solids were then dissolved in 75 ul DCM. 30 mg/mL benzyl alcohol solution was prepared in a separate vial, and 34 ul was added to the monomer solution (1 mg, 1 equivalent). 1 drop of DBU was added to start the polymerization. 3 hr later, 20.47 mg Boc-morpholinone (11 equivalent, 101.72 umol) was added as solid. 3 hr later, reaction was quenched by 6 drops of AcOH and transferred to a 2 kDA dialysis bag and dialyzed against MeOH for 4 hours. Dialyzed solution was concentrated to yield clear oil as the product. The block length (DP) and number average molecular weight (Mn) was determined by 1H-NMR end group analysis. Dispersity (D) was determined by gel permeation chromatography (GPC). Boc groups were removed to yield the deprotected oligomer as the active vehicle as follows. In a 1-dram vial, oligomers (0.5 μmol) were deprotected in a trifluoracetic acid/distilled DCM solution (1:4 v/v, 500 μL) under slow stirring and ambient atmosphere for 1 hour. Solvent was removed in vacuo and the samples were stored under high vacuum for 18 hours. The deprotected oligomer, as a thin film, was dissolved in DMSO to achieve 30 mM cation concentrations and stored at −20 C ready to use.

In general, the synthesis of the poly(alpha-amino ester) moiety is achieved through a ring-opening polymerization and/or copolymerization of morpholine-2-one and cyclic carbonate monomers. The N-Boc protected morpholinone (MBoc) polymerizes to high conversion (>85%), tunable Mn (1 kDa-20 kDa), and low molecular weight distributions (Mw/Mn-1.1-1.3) using an organocatalytic system. Post-polymerization deprotection of the Boc groups affords a cationic (diprotic, secondary amine) water-soluble polymer (−0.5M in D20, stable for >3 days). Furthermore, copolymerization of MBoc with MTC-dodecyl carbonate monomers followed by deprotection give rise to moderately charged cationic materials in high yield (>60%) with narrow polydispersity <1.4 PDI) and tunable block length. Block length is controlled by the ratio of initiator to monomer.

Copolymers or co-oligomers (block or statistical) can also be made by mixing two or more morpholin-2-one monomers, or by the copolymerization (or co-oligomerization) of one or multiple morpholin-2-one monomers with one or multiple cyclic carbonate monomers described herein. These carbonate monomers can incorporate a similar variety of side chain functionality, notably lipophilic groups or cationic groups to modulate oligonucleotide stability, delivery, and release properties. Furthermore, a variety of other commercially available cyclic ester monomers can be used including but not limited to lactide, glycolide, valerolactone, and/or caprolactone to incorporate lipophilic functionality.

Additional detailed methods of making the poly(alpha-amino ester) moiety are described in Blake et al., J. Am Chem Soc. 2014 136:9252-9255 and Blake et al., Chem Sci 2020 11:2951.

In some aspects, the compounds described here are compared to reference copolymers of cationic poly(alpha-amino ester) monomers and lipophilic monomers having a cyclic methyl trimethylene carbonate (MTC) polymer backbone. The reference compounds are described, for example, in WO 2018/022930 and WO 2020/097614.

### Compositions

Also provided are compositions, including pharmaceutical compositions, comprising a compound of formula I, or subformula thereof, and a carrier or excipient, including a pharmaceutically acceptable carrier or excipient.

In aspects, the composition or pharmaceutical composition comprises a compound of Formula I, or subformula thereof, non-covalently complexed with a nucleic acid or a plurality of different nucleic acids. The complexes condense to form nanoparticles ranging in size from about 100-400 nanometers (nm) in diameter or from about 150-300 nm, optionally when combined with nucleic acid at a representative charge ratio of 10:1 (cation:anion). In this context, size refers to the mean particle size (Z). Thus, the disclosure also provides compositions or pharmaceutical compositions comprising nanoparticles of a compound of Formula I non-covalently complexed with a nucleic acid or a plurality of different nucleic acids.

In general, a compound of Formula I is complexed with nucleic acid in an amount effective to produce a theoretical charge ratio of cationic compound to anionic nucleic acid of from about 4:1 to about 25:1 (cation:anion). In aspects, the theoretical charge ratio is 4:1, 5:1, 6:1, 8:1, 10:1, 15:1, 20:1, or 25:1. Theoretical (+/−) charge ratios are calculated as moles of cations to moles of phosphate anions, assuming full amine protonation, i.e., of the cationic nitrogen moieties of the poly(alpha-amino ester) monomer units, and phosphate deprotonation, i.e, of the nucleic acid. In some aspects, the charge ratio (+/−) is 5:1, 10:1, 20:1, or 25:1. Complexation of nucleic acid with a compound described herein may take place in the presence of a coordinating metal such as Zn⁺², Mg⁺², Ca⁺²; a dynamic non-covalent cross linker such as a carbohydrate; a counterion such as Cl⁻, AcO⁻, succinate, or citrate; or a solubility modulator such as a lipid or a polyethyleneglycol (PEG), or any combination thereof.

Also provided are compositions comprising nanoparticulate complexes of nucleic acid non-covalently attached to a compound of Formula I, or subformula thereof. In aspects, the charge ratio of copolymer and nucleic acid in the complex is about 5:1, 10:1, 20:1, or 25:1.

In accordance with the compounds and compositions described here, the nucleic acid may be an RNA or DNA. In aspects, the nucleic acid is messenger RNA (mRNA), small interference RNA (siRNA), short hairpin RNA (shRNA), micro RNA (miRNA), guide RNA (gRNA), CRISPR RNA (crRNA), transactivating RNA (tracrRNA), circular RNA (circRNA), self-amplifying RNA (saRNA), plasmid DNA (pDNA), minicircle DNA, and genomic DNA (gNDA), and combinations of two or more of any of the foregoing.

Also provided are pharmaceutical compositions comprising a compound of Formula I, or subformula thereof, as described herein, which can be used for therapy. In some aspects, the compound of Formula I, or subformula thereof, is complexed with a nucleic acid. In aspects, the composition has a compound of Formula I, or subformula thereof, but not a cargo nucleic acid. In accordance with these aspects, the cargo nucleic acid can be complexed with the compound of Formula I, or subformula thereof, before administration of the composition to a subject. In aspects, the nucleic acid is a therapeutic agent. In aspects, the nucleic acid encodes a therapeutic agent.

In some aspects, a composition can be a vaccine or a composition thereof, i.e. a composition that contains the vaccine and optionally a pharmaceutically acceptable carrier and/or immunological adjuvant. In aspects, the immunological adjuvant can include, but is not limited to, agonists of Toll-like Receptors (TLRs), agonists of the STING pathway, agonistic antibodies against CD40, OX40, CTLA4, PDI, or PDI-L, Freund's adjuvant, bryostatins, PKC modulators, and ligands for CD40, OX40, CD137, PD1, CTLA4 and any combinations thereof. In some aspects, the adjuvant can increase immunogenicity that is induced when a compound of Formula I, or subformula thereof, complexed with nucleic acid by co-administered with the complex to a subject.

The vaccine or vaccine composition can be used to prevent and/or treat a disease or condition or a pathogen associated with the disease or condition. In some aspects, the vaccine or vaccine composition contains a compound of Formula I, or subformula thereof, and a cargo nucleic acid. In some aspects, the compound of Formula I, or subformula thereof, complexed with nucleic acid, when administered to a subject, can induce an immune response, i.e. immunogenic. This immunogenicity can be induced, at least in part, when one or more antigenic peptides encoded by the cargo nucleic acid are expressed in the transfected cells.

The pharmaceutical compositions may contain pharmaceutically acceptable excipients or additives which may vary depending on the intended route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additional acceptable carriers, excipients, or stabilizers may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulation of the pharmaceutical compositions of the present disclosure can vary according to the route of administration selected (e.g., solution, emulsion).). Routes of administration can be, for example, intraperitoneal, intramuscular, subcutaneous, intravenous, intralymphatic, intraocular, retroorbital, intraural, intranasal, intratumoral, topical skin, topical conjunctival, oral, intravesical (bladder), intraanal and intravaginal.

In some aspects, the composition can include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In some aspects, the formulation is a controlled release formulation. The term “controlled release formulation” includes sustained release and time-release formulations. Controlled release formulations are well-known in the art. These include excipients that allow for sustained, periodic, pulse, or delayed release of the composition. Controlled release formulations include, without limitation, embedding of the composition into a matrix; enteric coatings; micro-encapsulation; gels and hydrogels; implants; and any other formulation that allows for controlled release of a composition.

### Methods of Use

The disclosure provides methods of targeted delivery of nucleic acids to particular types of cells and/or tissues in vitro, ex vivo, or in vivo. In aspects, the methods comprise contacting the target cell with a composition comprising nanoparticulate particles comprising a nucleic acid non-covalently attached to a a compound of Formula I, or subformula thereof. In aspects, the target cell is a B or T lymphocyte.

In aspects, the nucleic acid may be RNA or DNA. In aspects, the RNA is messenger RNA (mRNA), small interference RNA (siRNA), short hairpin RNA (shRNA), micro RNA (miRNA), guide RNA (gRNA), CRISPR RNA (crRNA), or transactivating RNA (tracrRNA). In aspects, the DNA is plasmid DNA (pDNA), minicircle DNA, or genomic DNA (gNDA).

In aspects, the nucleic acid may be a therapeutic nucleic acid or the nucleic acid may encode one or therapeutic agents, for example a cytokine, a T cell receptor (TCR), or a chimeric antigen receptor (CAR).

In some aspects, a method of transfecting a nucleic acid into a cell as described herein may be part of a method for gene editing or genetic engineering. For example, one or more nucleic acids may be transfected using the methods described herein in a CRISPR-based system or a transposon-based system for gene editing or genetic engineering. Accordingly, one or more nucleic acids may be transfected according to the methods described here, which nucleic acids may be located on one or more vectors. For example, the one or more nucleic acids may comprise a vector having a first nucleotide sequence encoding a CRISPR-Cas system guide RNA (gRNA) that hybridizes with a target sequence in the genome of a target cell and a second nucleotide sequence encoding a Cas9 protein. Alternatively, the gRNA and Cas9 protein can be located on different vectors, or either the gRNA or Cas9 protein may be produced in the target cell. The one or more nucleic acids may also comprise a CRISPR RNA (crRNA) and/or transactivating RNA (tracrRNA), each of which may be located on the same or a different vector as the gRNA and/or Cas9 encoding sequence.

In another aspect, the one or more nucleic acids may comprise a sequence encoding a transposase and a nucleic acid sequence of a gene of interest flanked by a transposase recognition site, which may be located on the same or different vectors.

In some aspects, a compound of Formula I may be complexed with one or more nucleic acids encoding one or more antigenic or immunogenic epitopes or peptides which may form a vaccine composition. For example, the one or more antigenic or immunogenic epitopes or peptides may be epitopes of a bacterial antigen, a viral antigen, or an antigen of a parasite. In some aspects, a mixture of two or more different compounds of Formula I may be complexed with one or more nucleic acids encoding one or more antigenic or immunogenic epitopes or peptides. Also provided are methods for preventing a disease or disorder, the method including administering a vaccine composition as described herein to a subject in need of therapy for the disease or disorder, optionally where administration is by a parenteral route, further optionally where administration is by an intravenous route, optionally where the subject is human. The method may also include where the disease or disorder is an infectious disease. The method may also include where the infectious disease is caused by an infectious agent, optionally where the infectious agent is a virus, a bacterium, or a protozoa.

In aspects, provided is a method for preventing an infectious disease or disorder, the method including administering a vaccine composition as described herein to a subject in need of therapy for the infectious disease or disorder, optionally where administration is by a parenteral route, further optionally where administration is by an intravenous route, optionally where the subject is human. In some aspects, the infectious disease or disorder is anthrax, caries, Chagas disease, a coronavirus infection, including a COVID19 infection, dengue, diphtheria, ehrlichiosis, hepatitis A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, or visceral leishmaniasis. In some aspects, the infectious disease or disorder is caused by caused by adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), SARS-CoV-2, rotavirus, Salmonella typhi, Staphylococcus aureus, Streptococcus pyogenes, varicella, West Nile virus, Yersinia pestis, or Zika virus.

In aspects, provided is a method of producing chimeric antigen receptor T cells (“CAR-T cells”), the methods comprising contacting isolated T cells with a composition comprising a a compound of Formula I, or subformula thereof non-covalently attached to a nucleic acid, wherein the nucleic acid is an mRNA encoding a chimeric antigen receptor (CAR) protein. In aspects, the T cells are CD8+ T cells or CD4+ T cells isolated from a human peripheral blood mononuclear cell (“PBMC”) fraction. In aspects, the mRNA encodes a chimeric antigen receptor protein that binds to an extracellular surface antigen, such as a cancer antigen, viral antigen, or bacterial antigen. In some aspects, the CAR protein binds to a tumor cell antigen selected from the group consisting of CD19, CD20, CD22, TAG72, B7-H3, MUC1, and MUC16.

In aspects, the methods comprise administering a composition comprising nanoparticulate particles of a nucleic acid non-covalently attached to a a compound of Formula I, or subformula thereof, including a pharmaceutical composition or vaccine composition, to a subject. Administration may be according to any suitable route, for example, parenteral, including e.g., intravenous, intramuscular, intradermal, subcutaneous, intraperitoneal, intracranial, etc., or transmucosal, including e.g., buccal, nasal, sublingual, transdermal, etc. In aspects, the subject is a mammal, for example a human, a non-human primate, a murine (i.e., mouse and rat), a canine, a feline, or an equine. In aspects, the subject is a human.

While various aspects and aspects of the present disclosure are shown and described herein, it will be obvious to those skilled in the art that such aspects and aspects are provided by way of example only. Numerous variations, changes, and substitutions will now occur to those skilled in the art without departing from the disclosure. It should be understood that various alternatives to the aspects of the disclosure described herein may be employed in practicing the disclosure.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “alkyl,” by itself or as part of another substituent, refers to an acyclic branched or unbranched hydrocarbon group containing from about 1 to 24 carbon atoms (C₁-C₂₄) or from 1 to 18 carbon atoms (C₁-C₁₈), which may be fully saturated, mono- or polyunsaturated and can include mono-, di- and multivalent radicals. Examples of saturated alkyl groups include methyl, ethyl, n-propyl, isopropyl, n-butyl, t-butyl, isobutyl, pentyl, hexyl, heptyl, octyl, nonyl, decyl, undecyl, dodecyl, octadecyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. In some aspects, an unsaturated alkyl group may be a linolenyl or oleyl. “Substituted alkyl” refers to alkyl substituted with one or more substituent groups, including where two hydrogen atoms from the same carbon atom in an alkyl substituent are replaced, such as in a carbonyl group. For example, a substituted alkyl group may include a carbonyl (—C(═O)—) moiety. The term “heteroalkyl” refers to an alkyl substituent in which at least one carbon atom is replaced with a heteroatom, as described in more detail below. If not otherwise indicated, the term “alkyl” includes unsubstituted, substituted, and/or heteroatom-containing alkyl substituents.

The term “alkoxy” refers to an alkyl group bound through a single, terminal ether linkage which may be represented as —O-alkyl, where alkyl is as defined above. Examples include methoxy, ethoxy, n-propoxy, isopropoxy, t-butyloxy, etc. The term “alkylthio” refers to a group —S-alkyl.

The term “heteroalkyl”, which is shorthand for “heteroatom-containing alkyl”, refers to an alkyl substituent in which at least one carbon atom is replaced with a heteroatom. Similarly, the term “heterocyclic” refers to a cyclic substituent that is heteroatom-containing, and the term “heteroaryl” refers to “aryl” substituents that are heteroatom-containing. In this context, “heteroatom-containing” refers to a molecule, linkage or substituent in which one or more carbon atoms are replaced with an atom other than carbon, such as nitrogen (N), oxygen (O), sulfur (S), phosphorus (P) or silicon (Si), and more typically in the context of the present disclosure with N, O, or S. Examples of heteroalkyl groups include alkoxyaryl, alkylsulfanyl-substituted alkyl, N-alkylated amino alkyl, and the like. Examples of heteroaryl groups are provided below.

In some aspects, the heteroatom is O, N, or S. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃, R—S—S—R′, and RO—S(O)ₓ—OR′. In some aspects, a heteroalkyl moiety may include one, two, three, four, or five heteroatom (e.g., O, N, S). In some aspects, a heteroalkyl moiety may include up to 8 optionally different heteroatoms (e.g., O, N, S).

The term “alkenyl” refers to a linear or branched hydrocarbon group of 2 to 24 carbon atoms containing at least one double bond, such as ethenyl, n-propenyl, isopropenyl, n-butenyl, isobutenyl, pentenyl, hexenyl, heptenyl, octenyl, nonenyl, decenyl, undecenyl, dodecenyl, octadecenyl, eicosenyl, and the like. In some aspects, alkenyl groups may contain 2 to about 18 carbon atoms (C₂-C₁₈) or 2 to 12 carbon atoms (C₂-C₁₂). The term “substituted alkenyl” refers to alkenyl substituted with one or more substituent groups, and the term “heteroalkenyl” refers to alkenyl in which at least one carbon atom is replaced with a heteroatom. A heteroalkenyl may optionally include more than one double bond and/or one or more triple bonds in additional to the one or more double bonds.

The term “alkylene” refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. In aspects, an alkylene substituent group may be methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—), octylene (—(CH₂)₈—) and the like. Similarly, the terms “alkenylene”, “alkynylene”, “arylene”, “aralkylene”, and “alkarylene” refer to di-radical alkenyl, alkynyl, aryl, aralkyl, and alkaryl groups, respectively. In some aspects, the aralkylene group is substituted on the alkylene moiety or the arylene moiety (e.g. at carbons 2, 3, 4, or 6) with a functional group. In the context of the present disclosure, alkylene groups are utilized as linking groups. Accordingly, these and other di-radical groups may be referred to herein as “linkers”, “linker groups”, “linker substituents”, “linking groups” or “linking substituents”. The alkylene, alkenylene, alkynylene, arylene, aralkylene, and alkarylene groups may also contain one or more functional groups. The term “functional group” in this context refers to di-radical moieties that contain one or more functional groups such as an oxo (—O—, such as in an ether linkage), amine (—NR—), carbonyl (—C(═O)—), carbonate, and the like. In some aspects, the functional group of a functional linker may be selected from oxo, —N₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl. In aspects, the alkylarylene is unsubstituted.

The term “amino” is used herein to refer to the group —NZ₁Z₂ wherein Z₁ and Z₂ are hydrogen or nonhydrogen substituents, with nonhydrogen substituents including, for example, alkyl, aryl, alkenyl, aralkyl, and substituted and/or heteroatom-containing variants thereof.

The terms “cycloalkyl” and “heterocycloalkyl” refer to cyclic versions of the “alkyl” and “heteroalkyl” groups defined above. Cycloalkyl and heterocycloalkyl are not aromatic. In aspects, a cycloalkyl group includes monocyclic hydrocarbon ring systems containing from 3 to 9 carbon atoms (C₃-C₉), one or more of which may be replaced with a heteroatom, and where such groups can be saturated or unsaturated, but not aromatic. The term “3 to 6 membered” in reference to a cycloalkyl or heterocycloalkyl refers to the number of atoms, i.e. carbon atoms or carbon and one or more heteroatoms, in the monocyclic ring. For example, the term “3 to 6 membered cycloalkyl or heterocycloalkyl” refers to cyclopropyl, cyclobutyl, cyclopentyl, cyclohexyl, heterocyclopropyl, heterocyclobutyl, heterocyclopentyl, and heterocyclohexyl. In aspects, a cycloalkyl group includes a bicyclic or multicyclic cycloalkyl ring system where multiple rings are fused together, where at least one of the fused rings is a cycloalkyl ring and the multiple rings are attached to the parent molecular moiety through any carbon atom contained within a cycloalkyl ring of the multiple rings. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Additionally, for heterocycloalkyl, a heteroatom can occupy the position at which the heterocycle is attached to the remainder of the molecule. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In aspects, the cycloalkyl is fully saturated. In aspects, the cycloalkyl is monounsaturated. In aspects, the cycloalkyl is polyunsaturated. In aspects, the heterocycloalkyl is fully saturated. In aspects, the heterocycloalkyl is monounsaturated. In aspects, the heterocycloalkyl is polyunsaturated.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” refers to cyclic groups that contain at least one aromatic ring, for example a single ring (e.g. phenyl) or multiple condensed rings (e.g. naphthyl). In some aspects of the present disclosure, the aryl group contains 6, 9 or 10 atoms such as phenyl, biphenyl, naphthyl, diphenylether, diphenylamine, benzophenone, indanyl, anthracenyl, 1,2-dihydronaphthyl, 1,2,3,4-tetrahydronaphthyl, fluorenyl, indanyl, indenyl and the like. A fused ring aryl refers to multiple rings fused together wherein at least one of the fused rings is an aryl ring and wherein the multiple rings are attached to the parent molecular moiety through any carbon atom contained within an aryl ring of the multiple rings. “Substituted aryl” refers to an aryl moiety substituted with one or more substituent groups, and the term “heteroaryl” refers to an aryl substituent in which at least one carbon atom is replaced with a heteroatom, as described in more detail below. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, where “alkyl” and “aryl” are as defined above. In general, aralkyl and alkaryl groups may contain from 6 to 30 carbon atoms. Aralkyl and alkaryl groups may, for example, contain 6 to 20 carbon atoms or from 6 to 12 carbon atoms.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents as described herein.

An “arylene” and a “heteroarylene,” alone or as part of another substituent, means a divalent radical derived from an aryl and heteroaryl, respectively. A heteroaryl group substituent may be —O— bonded to a ring heteroatom nitrogen.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The terms “halo” and “halogen” are used in the conventional sense to refer to a chloro, bromo, fluoro or iodo substituent.

The term “substituted” as in “substituted alkyl”, “substituted aryl”, and the like, refers to at least one hydrogen atom bound to a carbon or other atom that is replaced with one or more non-hydrogen substituents in the alkyl, aryl, or other moiety. The term “substituted or unsubstituted” preceding a list, as in “substituted or unsubstituted C₁-C₂₄ alkyl, C₁-C₂₄ heteroalkyl, cycloalkyl, heterocycloalkyl, aryl, heteroaryl, or alkylaryl” is intended to modify each member of the list, as in “substituted or unsubstituted C₁-C₂₄ alkyl, substituted or unsubstituted C₁-C₂₄ heteroalkyl, substituted or unsubstituted cycloalkyl, substituted or unsubstituted heterocycloalkyl, substituted or unsubstituted aryl, substituted or unsubstituted heteroaryl, or substituted or unsubstituted alkylaryl”. Examples of such substituents include, without limitation: functional groups such as halo, hydroxyl, sulfhydryl, C₁-C₂₄ alkoxy, C₂-C₂₄ alkenyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R is hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R is hydrogen, alkyl, aryl, alkaryl, etc.), arylimino (—CR═N(aryl), where R is hydrogen, alkyl, aryl, alkaryl, etc.), nitro (—NO₂), nitroso (—NO), sulfo (—SO₂—OH), sulfonato (—SO₂—O—), sulfonamide (—SO₂NH), C₁-C₂₄ alkylsulfanyl (—S-alkyl or “alkylthio”), arylsulfanyl (—S-aryl or “arylthiol”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl); and the hydrocarbyl moieties C₁-C₂₄ alkyl (including C₁-C₁₈ alkyl, C₁-C₁₂ alkyl, C₁-C₈alkyl and C₁-C₆ alkyl), C₂-C₂₄ alkenyl (including C₂-C₁₈ alkenyl, C₁-C₁₂ alkenyl, C₁-C₈ alkenyl, and C₁-C₆ alkenyl), C₅-C₃₀ aryl (including C₅-C₂₀ aryl, C₅-C₁₂ aryl), and C₆-C₃₀ aralkyl (including C₆-C₂₀ aralkyl, and C₆-C₁₂ aralkyl). In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated. In addition, the hydrocarbyl moieties may contain one or more heteroatoms, optionally N, O, or both.

It is understood that due to resonance a charge may be distributed across the molecule. The chemical structures and formulae set forth herein are constructed according to the standard rules of chemical valency known in the chemical arts, and as such one of skill in the art would recognize the equivalency of the moieties possessing resonance structures.

The suffix “ene” added on to any of the above groups means that the group is divalent, i.e. inserted between two other groups.

The symbol “ [symbol] ” denotes the point of attachment of a chemical moiety to the remainder of a molecule or chemical formula.

### EXAMPLES

Primary T lymphocytes are key players in immunology. Effective modulation of T lymphocytes by gene delivery is critical for various immunotherapies, such as chimeric antigen receptor T cell therapy (“CAR-T therapy”) and in vivo T cell reprogramming. However, efficient nucleic acid delivery to T lymphocytes has proven difficult. Currently, a widely used method in the laboratory setting is electroporation, but this method is difficult to scale, prone to cell killing, and unsuitable for many therapeutic applications. Viral vectors have been developed, but viral vehicles are limited by undesirable immunogenicity as well as the size of the nucleic acid “cargo” that can be accommodated. Lipid nanoparticle (LNP) systems represent another class of delivery vehicle developed for T lymphocytes. Exemplary LNP systems include the poly(alpha-amino ester) incorporated with T cell targeting antibodies such as anti-CD3, CD4, and CD5 to target T cells in vivo. This system provided a significant increase in T cell transfection in lymphoid organs such as the spleen, but the majority of transfected cells were in the liver. In addition, T cell depletion has been observed both in vitro and in vivo after treatment of CD3 conjugated LNPs, raising potential safety concerns. In some cases, LNPs have been used for T cell transfection without targeting ligands, such as LNP 93-017S, however, high doses of mRNA and repeated injections were needed to achieve suitable transfection efficiencies.

Polymeric systems represent another class of nucleic acid delivery system that is being developed for T cell transfection. For example, a comb-shaped polymer, CP-25-16, demonstrated a 25% transfection of primary human T cells in vitro and a poly(beta-amido ester), PBAE-447, system formulated with an anti-CD3 antibody was reported to transfect 80% T cells in vitro and 8% splenic T cells in vivo.

Realizing the full potential of nucleic acid-based technologies requires the development of delivery systems that are able to efficiently complex, protect, deliver, and release nucleic acids into cells. This is especially true for mRNA, which is unstable in physiological environments and can barely enter the cytoplasm of target cells. Delivery systems for mRNA must therefore be able to protect the molecule during transport to target cells. In addition, the delivery vehicle must not only efficiently deliver the mRNA cargo across the cell membrane into the cytoplasm but it must also rapidly release the mRNA for translation. Effective mRNA delivery systems are critical for dRNA therapies, including vaccines. Yet, despite considerable effort, delivering RNA, in particular mRNA, to a desired cell type with high-efficiency remains challenging.

The present invention addresses the need for improved polymeric nucleic acid delivery vehicles and demonstrates their efficacy in a particularly difficult cell model system, T lymphocytes, as a proof of concept of their general utility as nucleic acid transporters, as described in more detail below.

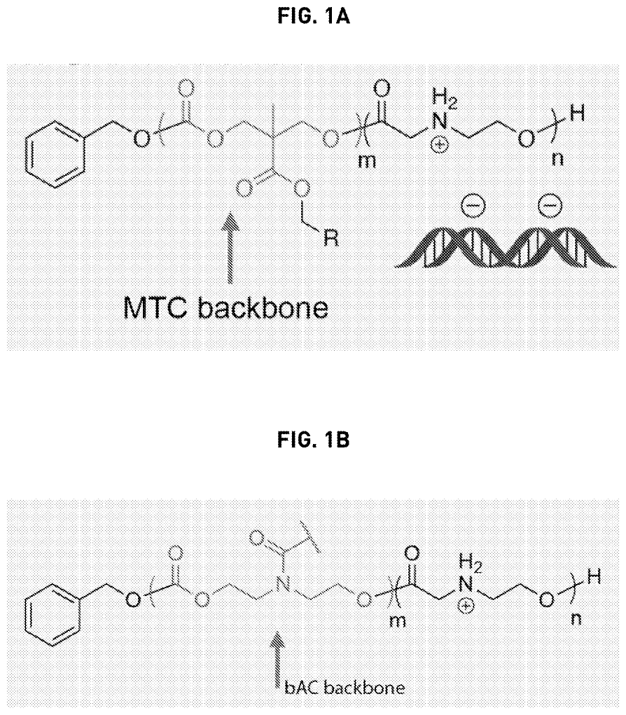

Charge-altering releasable transporters (CART) are a class of polymeric nucleic acid delivery systems previously developed to deliver nucleic acid cargoes to cells for use in various biomedical applications. CARTs are block or statistical oligomers consisting of initiators, lipophilic blocks and polycationic blocks prepared by organocatalytic ring-opening polymerization (OROP). FIG. 1A shows a schematic representation of a generic CART structure with a methyl-trimethylene carbonate backbone (MTC).

The present invention provides a new CART system including a different polymeric backbone, namely a beta-amido carbonate backbone (bAC), and a different spacing of components, as shown in FIG. 1B. As discussed in more detail below, compared to MTC-based CARTs of similar structure, the bAC CARTs described here showed significantly improved transfection efficiencies, which was due to increased nucleic acid uptake.

Many polymeric backbones have been derived from 6- or 7-membered cyclic ester and carbonate monomers due to ease of synthesis and favorable thermodynamics for polymerization, Here, an 8 membered cyclic ester, 6-acetyl-1,3,6-dioxazocan-2-one, was selected following a screen of various reagents. The present inventors believed that the altered backbone structure may provide improved properties arising from, for example, changes in the polymer flexibility and/or the location of functional attachments. As schematically illustrated in FIG. 2A, the cyclic ester monomer was functionalized with a lauryl lipid, to form a bAC monomer, referred to herein as “bAC-A”. As shown in the figure, the functionalized diol was obtained in quantitative yield by coupling lauroyl chloride with diethanolamine. By screening various cyclization reagents and conditions, yields of 45-50% were obtained using a slow triphosgene addition in diluted diol reactants. Next, as illustrated in FIG. 2B, bAC-A was copolymerized with Boc protected morpholinone blocks, with benzyl alcohol as the initiator using the organocatalytic ring opening polymerization (OROP) methodology as previously described in Blake et al., J. Am Chem Soc. 2014 136:9252-9255 and Blake et al., Chem Sci 2020 11:2951. Following TFA deprotection, the resulting bAC CARTs, designated “bAC-1a” contained about 17 dodecyl lipid units and 10 cationic poly(alpha-amino ester) units.

Transfection performance was evaluated as compared to two reference CARTs, an MTC analog of bAC-1a (D15A11) and a mixed lipid CART that had demonstrated efficient transfection of a T lymphocyte cell line, Jurkat cells, in previous studies, referred to as Oleyl-Nonenyl Amino CART, or “ONA” CART. Briefly, the different CARTs were complexed with 200 nanograms (ng) of enhanced green fluorescent protein (EGFP) mRNA and transfected into Jurkat cells. Briefly, 100 ng mRNA was first dissolved in PBS pH 5.5. CARTs in DMSO solution were then added and mixed by a micropipette for 20 seconds. The formulations were immediately added to cells. Transfection performance was evaluated by flow cytometry 24 hr after transfection. As shown in FIG. 2C, compared to the two reference CARTs, bAC-1a yielded >70% eGFP transfected cells, significantly outperforming both the D15A11 CART and the ONA CART.

Next, seven additional bAC monomers (bAC-B to H) functionalize with different lipid moieties as shown in Table 2 and FIG. 2D, were synthesized. bAC monomers A-H refer to the cyclic ester plus lipid moiety, e.g., bAC-A exemplified in FIG. 2A. The copolymers were synthesized as shown in FIG. 2B and are designated bAC-1 thru bAC-8. Each was polymerized to three different block lengths, a-c, as shown in the table below and FIG. 2D. The lipid moieties included straight chain and unsaturated lipids (bAC-A to C), an isoprenoid lipid (bAC-D), and four branched lipids (bAC-E to H).

TABLE 2
Lipid structures of bAC monomers used to prepare representative compounds of Formula Ia, bAC-1 thru bAC-7, having different
monomer lengths (z₁, z₃), designated a, b, or c as shown in the table.

| Monomer | Lipid Structure | # | a | b | c |
|---|---|---|---|---|---|
| A | 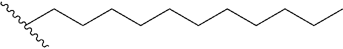 [SMILES: *CCCCCCCCCCC] | 1 | 17:10 | 10:10 | 10:16 |
| B | 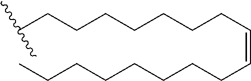 [SMILES: *CCCCCCC/C=C\CCCCCCCC] | 2 | 16:8 | 10:10 | 10:17 |
| C | 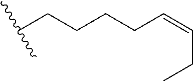 [SMILES: *CCCC/C=C\CC] | 3 | 17:10 | 11:10 | 11:17 |
| D | 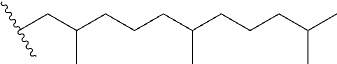 [SMILES: *CC(C)CCCC(C)CCCC(C)C] | 4 | 17:10 | 10:11 | 9:16 |
| E | 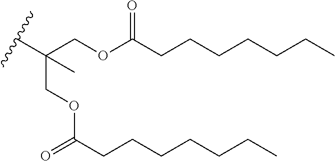 [SMILES: *C(C)(COC(=O)CCCCCCC)COC(=O)CCCCCCC] | 5 | 19:9 | 10:9 | 10:14 |
| F | 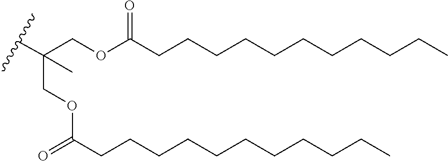 [SMILES: *C(C)(COC(=O)CCCCCCCCCCC)COC(=O)CCCCCCCCCCC] | 6 | 19:10 | 10:9 | 8:18 |
| G | 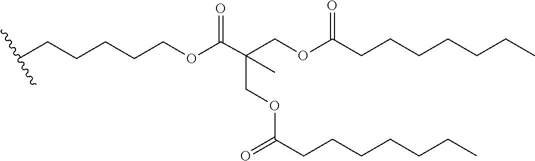 [SMILES: *CCCCCOC(=O)C(C)(COC(=O)CCCCCCC)COC(=O)CCCCCCC] | 7 | 17:10 | 10:10 | 12:18 |
| H | 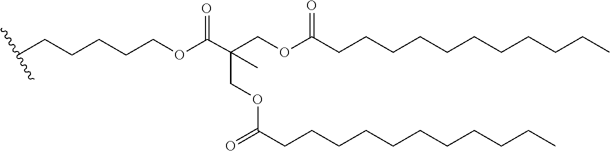 [SMILES: *CCCCCOC(=O)C(C)(COC(=O)CCCCCCCCCCC)COC(=O)CCCCCCCCCCC] | 8 | 16:9 | 10:9 | 8:18 |

In Table 2, the monomer has a structure of

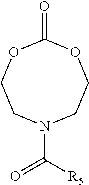

where R5 is Lipid 1, Lipid 2, Lipid 3, Lipid 4, Lipid 5, Lipid 6, Lipid 7, or Lipid 8.

Each of the bAC monomers were co-polymerized as described above into three different monomer lengths, as determined by 1H NMR. For example, bAC-1a contains ~17 bAC units and ~10 cationic AAE units, bAC-1b contains ~10 bAC units and ~10 AAE units, and bAC-1c contains ~10 bAC units and ~17 AAE units.

In addition, four reference polymers having MTC backbones with the same lipid moieties as the bAC compounds were prepared, for comparison. FIG. 2E shows the structures of the MTC compounds (referred to in the figure as “MTC CARTs”), including the monomers, A, D, G, and H, the lipid moieties, and the resulting polymers, MTC-1b, MTC-4b, MTC-7b, and MTC-8b.

All compounds were prepared out of glove box using standard Schlenk link techniques, as described above (see representative procedure to synthesize Boc-protected bAC-1 a, supra).

The poly(alpha-amino ester) (AAE) monomer units of the compounds electrostatically associates with nucleic acids at acidic pH (<5.5) to form nanoparticle complexes. Nanoparticle size was evaluated by dynamic light scattering (DLS). Compounds were added to PBS 5.5 containing 420 ng of EGFP mRNA (Trilink) at 10:1 charge ratio (cation:anion). The formulations were mixed for 20 seconds using a micropipette (drawing and dispensing 100 ul twice/second) before administration to mice via retro-orbital injections. The formulations were then immediately transferred to a disposable clear plastic cuvette, and the sizes measured by NanoBrook Omni (Serial No: 280097). Surface charge was monitored at 0, 10, 20 min after formulation using the same instruments. bAC/mRNA complexes formed particles ranging from 130 to 280 nm, with no apparent trend across lipid type or block length. Compared to MTC compounds, bAC compounds formed mRNA complexes of similar size except for bAC-8b (FIG. 2F). A distinct biophysical trait of the “CART” technology is decreasing surface charge over time caused by 0-N acyl shift. The surface charge of bAC compounds was monitored as zeta potential, which showed a similar trend compared with a reference MTC compound, referred to as “ONA CART”.

bAC compounds were evaluated as nucleic acid transfection reagents in Jurkat cells as described above but using a lower amount of mRNA (100 ng). Briefly, Jurkat cells were acquired from ATCC and maintained in suspension culture with RPMI 1640 medium supplemented with 2 mM glutamine, 10% FBS, and 1% penicillin-streptomycin at 37° C. in a humidified incubator with 5% CO2, and regularly tested for mycoplasma contamination (Lonza LT07-318). Cells were subcultured when cell concentration reached 8×10⁵ cells/mL. Before transfection Jurkat cells were washed twice with serum-free media and resuspended in serum-free medium at 4×10⁶ cells/ml, 25 ul of cell suspension were added into a 96-well round bottom plate (100,000 T cell per well). CART transfection was performed as described above, using 100 ng of mRNA per condition. After 2-4 hours, 150 ul of complete media was added to each well. Flow cytometry was performed 24 hours after transfection. Top performers bAC-4b, bAC-4c, and bAC-7b were identified, reaching a maximum of 73% EGFP+ cells (FIG. 2G). Transfection performance of CARTs was also evaluated by integrating fluorescence intensity of all EGFP+ cells (here termed AUC), which indicated the strength of protein expression. Measured as AUC, the bAC compounds also outperformed the reference MTC compound, ONA CART, indicating that bAC compounds are potent nucleic acid transporters for Jurkat cell delivery. All bAC compounds generated minimal toxicity during transfection.

Confocal microscopy was used to track Cy3-labeled mRNA delivered into Jurkat cells by the bAC compounds. The results for a representative compound, bAC-7c, showed notably more localized mRNA distribution compared to the reference MTC compounds, ONA CART and MTC-7c.

While the Jurkat cell line is commonly used as a proximate model to evaluate T cell transfection efficacy due to its ease of access and maintenance, effective transfection of primary T lymphocytes is needed for translation to clinical applications. Accordingly, we evaluated the performance of the bAC compounds in primary human T lymphocytes. Human peripheral blood mononuclear cells (PBMC) were isolated from whole blood by density gradient centrifugation using SepMate tubes (StemCell Technologies) with Lymphoprep following the manufacturer's instructions. PBMC's were counted and resuspended in CryoStor CS10 freezing medium (StemCell Technologies) at 10×10⁶ cells/ml for long-term storage in liquid nitrogen. PBMCs were thawed and counted on the day of transfection to isolate total human T cells using a Pan T cell isolation kit, human, according to the manufacturer's instructions (Miltenyi Biotec). Isolated T cells were counted and activated with Dynabeads Human T-Activator CD3/CD28, according to the manufacturer's instructions (Gibco). Briefly, in a 24-well plate, isolated T-cells were activated with 25 ul of Dynabeads for every 1×10⁶ T cells in 1 ml of complete medium (RPMI 1640 medium containing 10% FBS, 1% penicillin/streptomycin, 50 μM β-mercaptoethanol, and 1% L-glutamine). After 6 hours in culture, Dynabeads were removed using the DynaMag magnet and T cells were washed twice with serum-free RPMI medium. After counting, cells were resuspended in serum-free medium at 4×10⁶ cells/ml, and 25 ul of cell suspension were added into a 96-well round bottom plate (100,000 T cell per well). Transfection was performed as described above, using 100 ng of mRNA per condition. After 2-4 hours, 150 ul of complete media was added to each well. Flow cytometry was performed 24 hours after transfection.

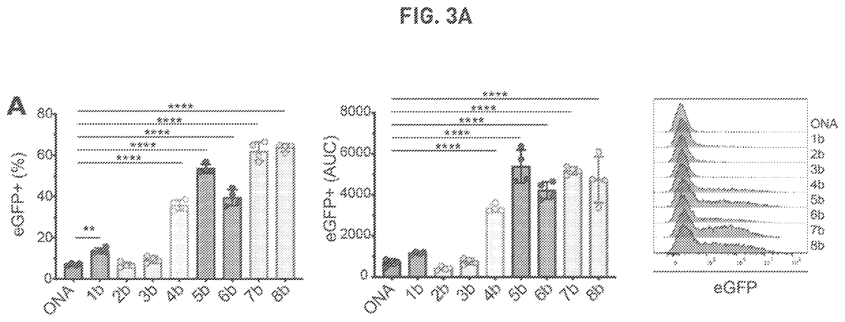

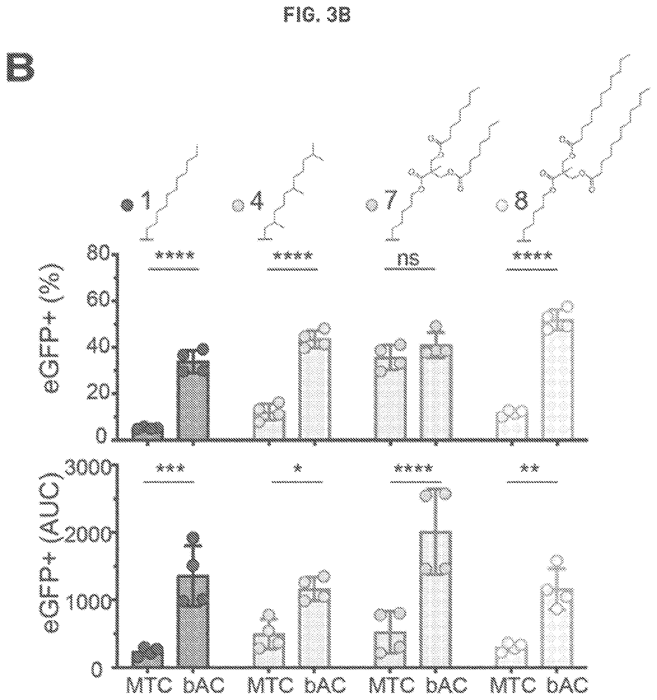

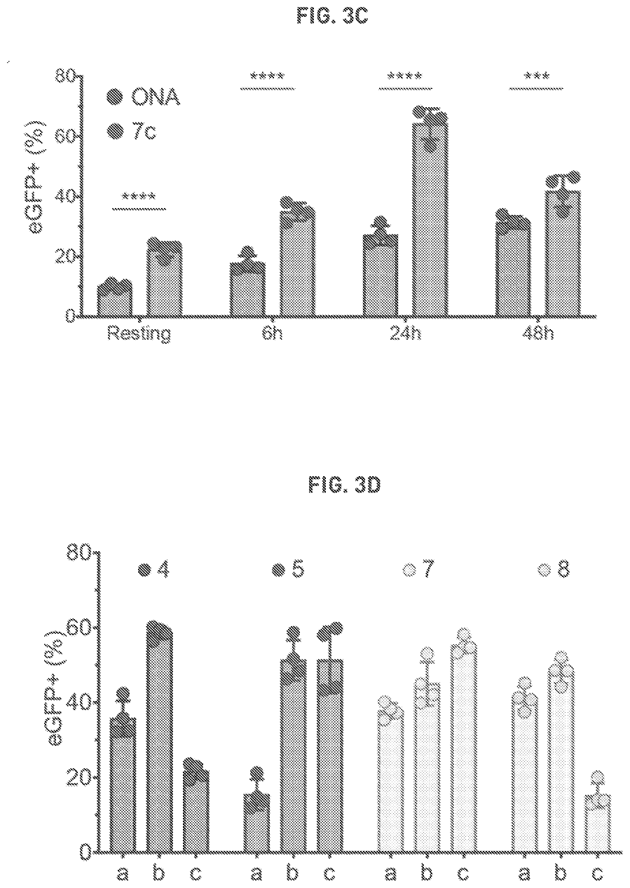

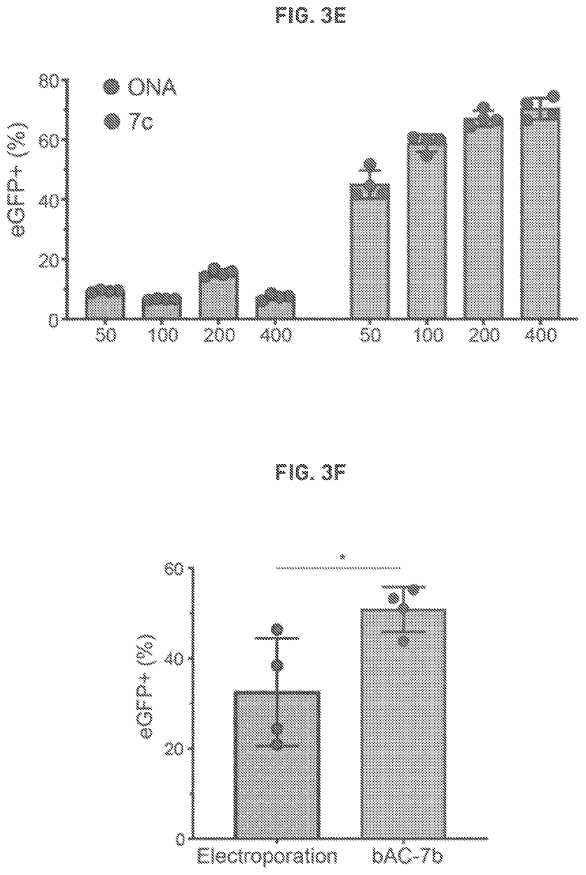

Delivery of EGFP mRNA to the human T lymphocytes isolated from PBMC was evaluated for each of bAC compounds 1b-8b (FIG. 3A). Compared to the reference MTC compound, ONA CART, which transfects about 10% of cells, bAC-7b transfected up to 65% of cells and several other bAC CARTs achieved greater than 40% transfection. bAC-7b resulted in 5-fold AUC over ONA, indicating the amount of protein expression was also increased. To assess whether the improved nucleic acid delivery and expression was primarily effected by differences in lipid attachment or differences in the polymeric backbone, each of the bAC compounds bAC-1b, 4b, 7b, and 8b was compared to its respective MTC analog, MTC-1b, 4b, 7b, and 8b. As shown in FIG. 3B, bAC-1b, 4b, and 8b significantly outperformed their MTC analogs, e.g. bAC-1b vs MTC-1b, in terms of number of EGFP+ cells in primary human T lymphocytes. The AUC of bAC compounds also outperformed all MTC analogs, indicating that the bAC backbone itself is responsible for a significant amount of the observed improvement in primary T cell transfection, irrespective of the lipid moiety utilized.

Activation is required for efficient T lymphocyte transfection. Methods and time of cell activation may significantly impact delivery efficacy. Various T cell activation reagents were tested and Dynabeads Human T-Activator CD3/CD28 was selected for experiments to optimize the time of transfection following T cell activation. As shown in FIG. 3C, both ONA and bAC-7c compounds exhibited a time-dependent mRNA delivery efficacy. In addition, bAC-7c significantly outperformed ONA at every time of T cell activation tested, achieving a peak of expression after 24 h of activation (65% of EGFP+ cells). Interestingly, bAC-7c was also able to transfect unactivated T cells with an efficacy greater than 25% (measured as percentage of EGFP expressing cells).

Using the optimized activation procedure, mRNA delivery efficacy was evaluated for the four highest performing bAC compounds having four different lipid moieties, bAC-4, bAC-5, bAC-7, and bAC-8, and varying monomer lengths, designated a, b, and c (see Table 1 for detail). The results are shown in FIG. 3D. All bAC compounds performed well in block length “b” while block length “a” resulted in relatively decreased transfection and transfection efficiency of block length “c” bAC compounds varied with the various lipid moieties. Three compounds, bAC-4b, bAC-5b, and bAC-7c, were selected for further optimization of charge ratios (N to P) and a 10:1 charge ratio was selected.

The best performing compound, bAC-7c was evaluated for transfection efficiency with different amounts of mRNA. As shown in FIG. 3E, 50% transfection was achieved with as little as 50 ng mRNA. Transfection efficiency reached a maximum of about 70% between 200 ng and 400 ng mRNA. No cell toxicity was observed at any dose.

Compared to electroporation, which is a standard process used in ex vivo transfection, bAC-7b provided higher protein expression measured as either percentage of EGFP+ cells or as AUC (FIG. 3F). By this measure, bAC-7b outperformed electroporation by 5-fold, indicating that with the same level of transfected cells, mRNA is being translated at higher rates after mRNA delivery with the bAC compound. Both delivery methods maintained good cell viability after treatment.

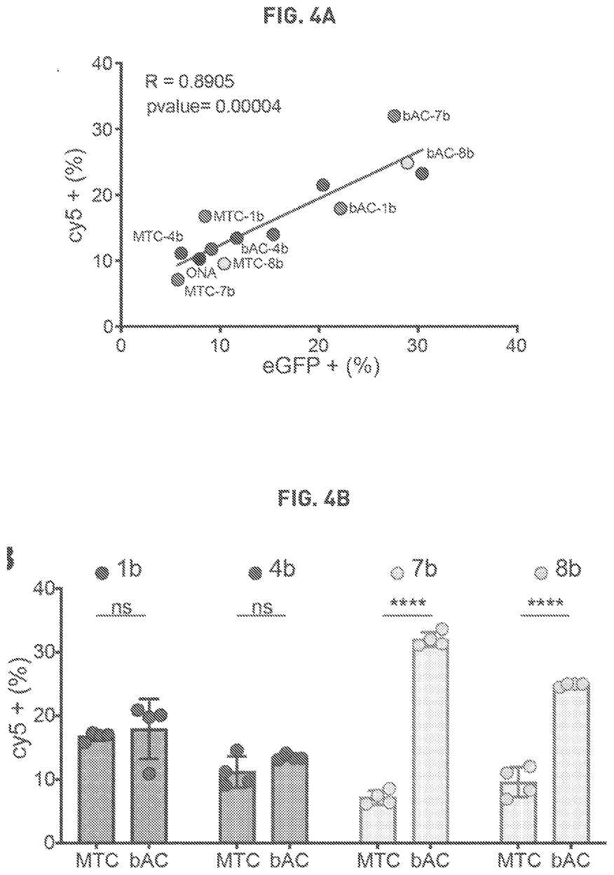

To evaluate the mechanisms behind the improved mRNA delivery observed with the bAC compounds, a fluorescently labeled mRNA complexed with representative bAC compounds was used to simultaneously measure cellular uptake and protein translation. Cellular uptake, measured as cy5+ cells, and protein translation, measured as EGFP+ cells, were positively correlated (Person correlation=0.8905, p=0.00004) (FIG. 4A). Thus, most of the mRNA delivered into cells by the bAC compounds is efficiently internalized, remains stable, and can escape the endosomal compartment to be available for translation in the cytosol. Two of the bAC compounds, bAC-7b and bAC-8b also showed a significant increase in cellular uptake (measured as % cy5+ cells) compared to their MTC analogs (FIG. 4B).

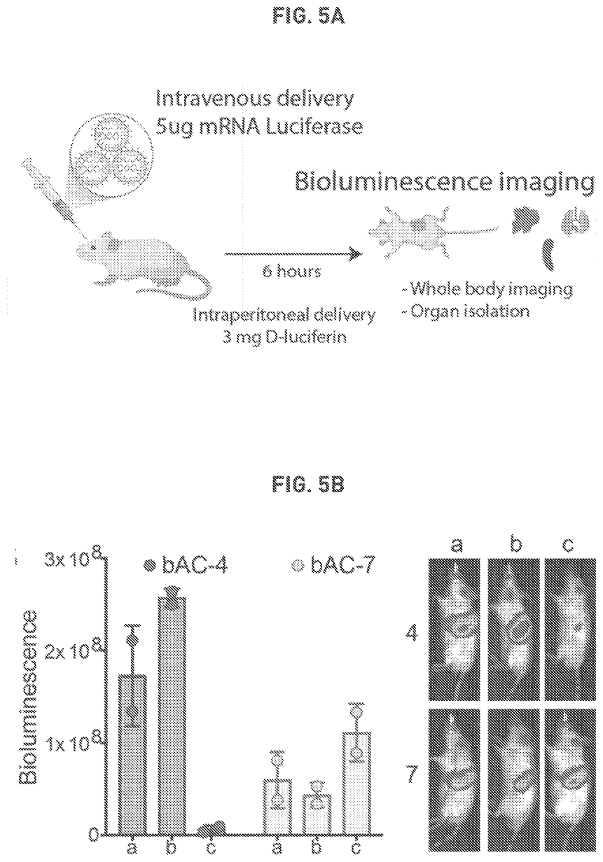

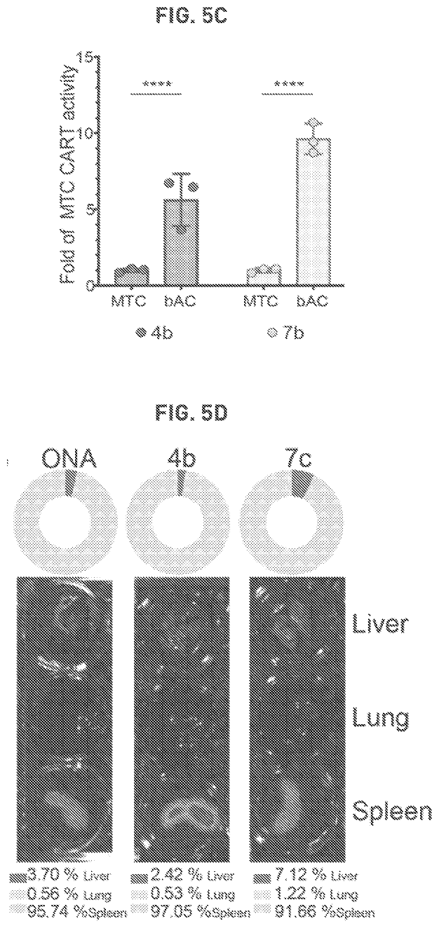

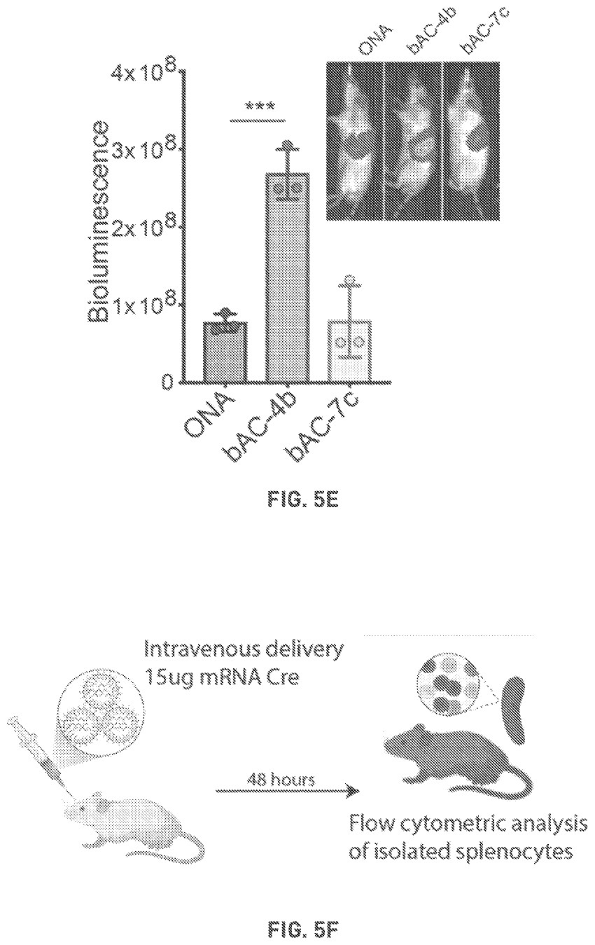

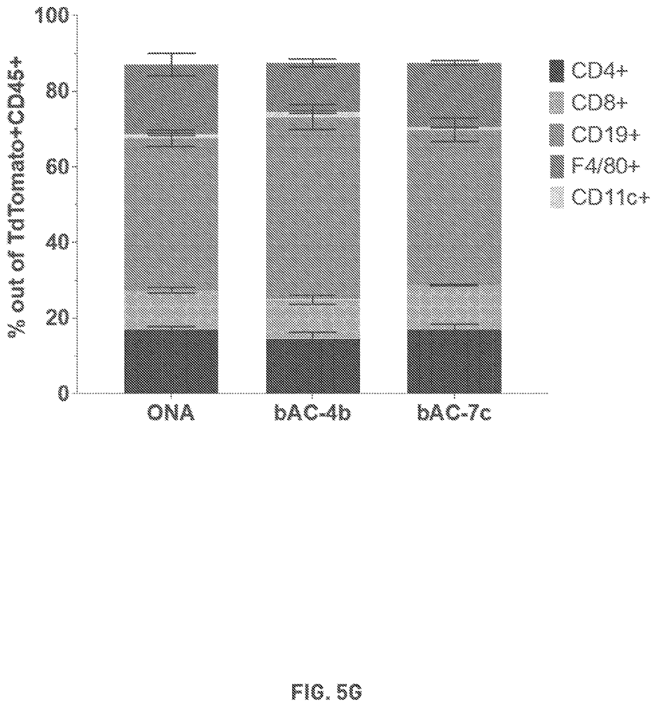

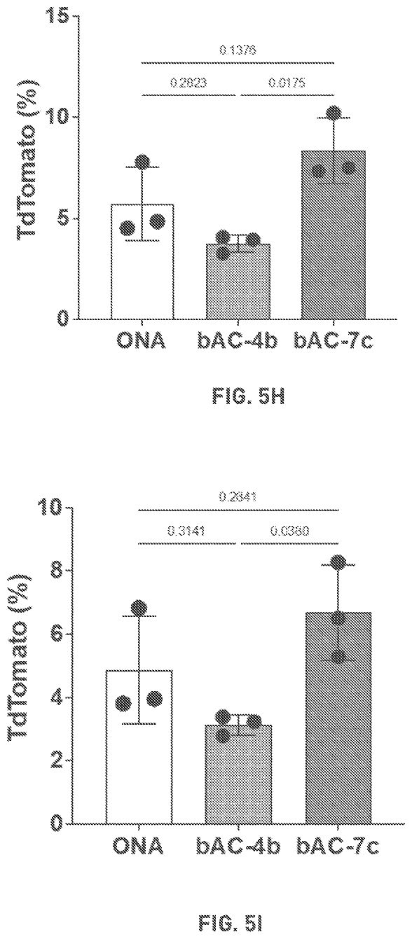

Next, the ability of bAC compounds to transfect T cells in vivo was evaluated, since this is an important benchmark for clinical translation. Six representative bAC compounds, bAC-4a thru 4c, and bAC-7a thru 7c, were complexed with luciferase mRNA and the complexes delivered intravenously to BALB/c mice. The protocol is depicted schematically in FIG. 5A. Briefly, Female BALB/c mice were purchased from Jackson Laboratory and housed in the Laboratory Animal Facility of the Stanford University Medical Center. CARTs were complexed with 5 g luciferase mRNA (Trilink) in PBS 5.5 to a total volume of 100 μl at 10:1 N/P ratio, with the same formulation procedure described above. The formulations were mixed for 20 seconds using a micropipette (drawing and dispensing 100 ul twice/second) before retro-orbital injections. 6 hr after injection, firefly luciferin solution (100 μl, 30 mg/mL) was injected intraperitoneally, and luminescence was measured using AMI Imaging system CCD camera and analyzed with Aura.

After administration of D-luciferin, luciferase protein expression was evaluated 6 hr post-injection by whole-body imaging with a CCD camera. As shown in FIG. 5B, bAC-4b had a stronger luciferase signal than bAC-4a and bAC-4c, while bAC-7c outperformed bAC-7a and bAC-7b. This performance was consistent with that observed in primary T lymphocytes for these compounds. However, in vivo luciferase expression for bAC-4b delivery was 2.5-fold higher than that of bAC-7c, even though bAC-7c slightly outperformed bAC-4b in vitro. The effect of charge ratios was evaluated for bAC-4b and bAC-7c and a 10:1 charge ratio was selected as optimal. As shown in FIG. 5C, both bAC-4b and bAC-7b significantly outperformed their MTC analogs. In addition, as shown in FIG. 5D and FIG. 5E, bAC compound transfected cells were located primarily in the spleen. In particular, both bAC-4b and bAC-7c showed excellent selectivity (>90%) to the spleen, which is highly favorable for in vivo T cell transfection.

To further study the cell specificity of in vivo mRNA delivery using the bAC compounds, a Cre recombinase murine model (Ai14 mice) was used to measure specific cell recombination after i.v. delivery of mRNA Cre complexed with bAC compounds (FIG. 5F). In this mouse model, cells that internalize mRNA and efficiently translate the Cre recombinase protein are detectable by their expression of the fluorescent protein tdTomato following Cre-mediated recombination. Briefly, female Ai14 mice were purchased from Jackson Laboratory and housed in the Laboratory Animal Facility of the Stanford University Medical Center. Compounds were complexed with 15 μg Cre mRNA (Trilink) in PBS pH 5.5 to a total volume of 100 μl at 10:1 N/P ratio, as described above. The formulations were mixed for 20 seconds using a micropipette (drawing and dispensing 100 ul twice/second) before retro-orbital injections. Spleens were isolated 48 h after transfection to measure Cre-mediated recombination. Briefly, spleens were smashed with a 100-μm strainer to make a single-cell suspension. Red blood cells were lysed before staining. Single-cell samples were then stained with Zombie NIR (BUV570, BioLegend 423103), anti-CD45 (clone 145-2C11, BioLegend), anti-CD8a (clone 53-6.7, BioLegend), anti-CD4 (clone RM4-5, BioLegend), anti-CD11c (clone IM7, BioLegend), anti-CD19 (clone 30-F11, BioLegend), anti-F4/80 (clone H1.2F3, BioLegend). Stained cells were washed twice and analyzed by flow cytometry.

After i.v. delivery of 15 μg of mRNA Cre in Ai14 mice, Cre-mediated recombination (tdTomato+) was observed in most of the CD45+ leukocytes in the spleen, including T cells (CD4+ and CD8+) and antigen-presenting cells such as dendritic cells (CD11c+), macrophages (F4/80+), and B cells (CD19+). As expected, most of the recombined cells were B cells (40-50%), which represent >55% of cells in the mouse spleen. No significant difference in cell proportions was observed between bAC-4b and bAC-7c (FIG. 5G). Importantly, up to 8% Cre-mediated recombination was achieved in CD4+ and CD8+ T cells in the spleen with bAC-7c CART without using a targeting ligands. Interestingly, bAC-7c resulted in a 2-fold increase in T cell transfection compared to bAC-4b (see FIG. 5H, CD4+ T cells, and FIG. 5I, CD8+ T cells), even though bAC-4b resulted in higher luciferase activity. One hypothesis is that the A14 model provides information only on the percentage of cells transfected and not protein expression levels. It is possible that bAC-4b transfects a smaller percentage of cells but results in more protein expression.

In summary, these experiments showed that bAC CARTs can mediate efficient mRNA delivery to T lymphocytes in vivo and can effectively deliver mRNA to the spleen without the use of targeting ligands.

A proof of concept experiment was performed in order to demonstrate the therapeutic potential of bAC compounds for CAR-T therapy. In preliminary experiments, the state of activation and functionality of T cells after transfection with bAC complexed mRNA was assessed by measuring the levels of the activation markers, CD25 and CD69, and the exhaustion marker PD1. There was no difference in phenotype and activation state between bAC transfected T cells and untreated controls (data not shown). In addition, the capacity of transfected T cells to produce effector cytokines IFN-γ and TNF-α after 24 h stimulation with Dynabeads Human T-Activator CD3/CD28 was assessed. No significant differences between bAC-7c and untreated cells was observed (data not shown). Finally, the T cell proliferation potential after non-specific activation with anti-CD3/CD28 and the cytokine IL-2 was assessed. Here too there were no significant differences in cell growth and viability after bAC transfection compared to untreated cells (data not shown). These data demonstrate the maintenance of phenotype and functionality of human T cells after transfection with a representative bAC compound, bAC-7c.

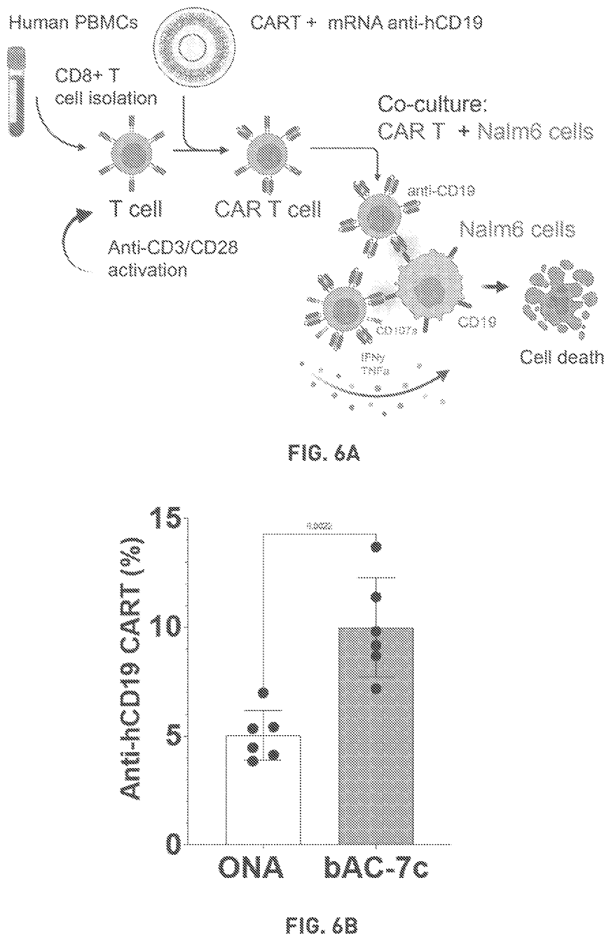

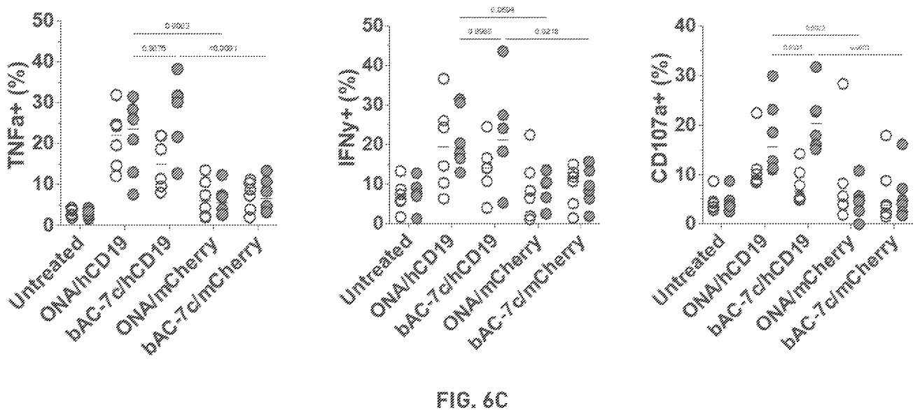

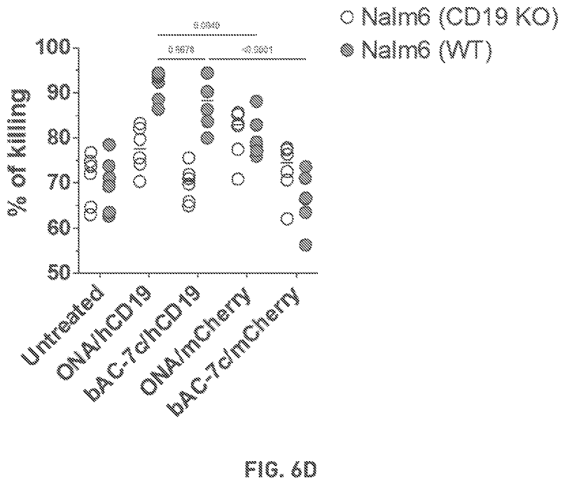

In order to test the ability of bAC complexed mRNA to enable ex vivo generation of highly cytotoxic CAR-T cells, activated CD8+ T cells were transfected with anti-human CD19 (hCD19) mRNA and the function of the resulting anti-hCD19 CAR T cells was assessed by co-culturing with Nalm6-GL. Nalm6-GL is a B cell precursor leukemia cell line that stably expresses GFP and firefly luciferase. FIG. 6A shows a schematic representation of the assay used to assess generation of cytotoxic CAR-T cells. CD8+ T cells were transfected with either the reference ONA CART or bAC-7c, each complexed with anti-human CD19 CAR mRNA. Transfected cells were assessed for antigen specific cytotoxicity and functional phenotype 20 hours after transfection.

As shown in FIG. 6B the percentage of anti-hCD19 expression 20 hours post-transfection was significantly higher for bAC-7c transfected cells, compared cells transfected with the reference ONA CART compound.

To assess the impact of anti-hCD19 CAR on CD8+ T cell function, levels of CD107a, an indicator of degranulation activity, and expression of cytokines IFN-γ and TNF-α were analyzed in transfected and untransfected CD8+ T cells during co-culture with Nalm6-GL cells at an E:T ratio of 1:4. FIG. 6C shows the percentage of cells expressing TNF-α, IFN-γ and CD107a. Compared to control cells, which were untransfected CD8+ T cells and cells transfected with mCherry mRNA, CD8+ T cells transfected with anti-hCD19 (using either ONA or bAC-7c) showed a higher frequency of CD107a expression and an increased frequency of IFN-γ and TNF-α expression, indicated elevated CD8+T activation and function due to CAR expression.

Next, the cytotoxic activity of CAR-transfected CD8+ T cells was evaluated. Wild-type or CD19 knock-out Nalm6-GL cells were co-cultured with CAR T cells transfected with ONA/hCD19 or bAC-7c/hCD19 at 10:1 effector-to-target (E:T) ratio. FIG. 6D shows the percentage of cell killing by CAR transfected cells and controls, untransfected (“untreated”) cells and ONA/mCherry or bAC-2/mCherry transfected cells. As illustrated in the figure, anti-hCD19 CAR T cells showed significantly increased killing of Nalm6-GL cells after 13 hours compared to untransfected cells or cells transfected with mCherry mRNA. In contrast, co-culturing with anti-hCD19 CAR T cells did not lead to increased killing of CD19 knock-out Nalm6-GL cells, indicating the dependence of Nalm6-GL cell killing by anti-hCD19 CAR T cells on CD19 expression. Overall, these findings demonstrate that bAC-7c transfection enables high expression of anti-hCD19 CAR in CD+8 T cells, leading to specific targeting of Nalm6 leukemia cells through CD19-CAR interaction.

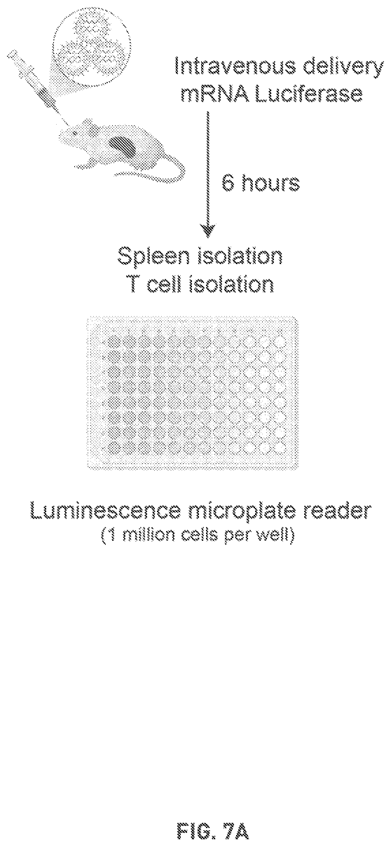

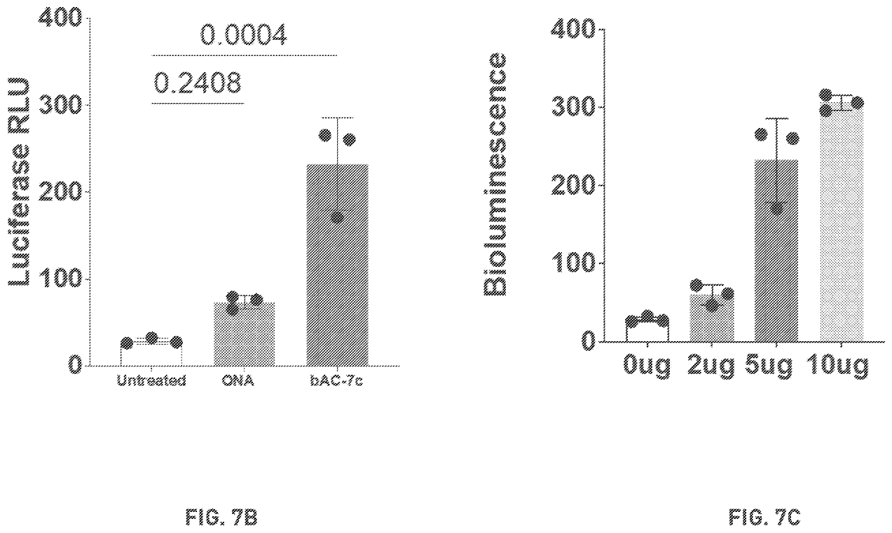

Since the A14 model provides information only on the percentage of cells transfected and not protein expression levels, a different assay was used to measure protein translation directly in T cells after in vivo delivery of mRNA with bAC or ONA compounds. The experiment is depicted graphically in FIG. 7A. As shown in the figure, luciferase mRNA was injected intra-orbitally followed 15 hours later by isolation of T cells and measurement of luciferase expression in a plate format.

As shown in FIG. 7B, delivery of mRNA luciferase with bAC-7c demonstrated a 3-fold increase in luciferase expression in splenic T cells compared to ONA CART. These data indicate that bAC-7c induces more protein expression in splenic T cells than the reference compound, ONA CART.

We also tested different doses (2, 5, 10 ug) of luciferase mRNA complexed with bAC-7c and observed dose-dependent luciferase activity in splenic T cells, indicating transfection could be boosted with a higher mRNA dose (FIG. 7C). These doses are comparable to those used clinically (e.g. 6 ug mRNA for a 20 g mouse, corresponding to 0.3 mg/kg).

To determine the intra-spleen biodistribution of mRNA after delivery with bAC-7c, mRNA covalently labeled with Cy5 was utilized. Briefly, 2 hours after i.v delivery of Cy5-mRNA with bAC-7c, spleens were isolated and the levels of Cy5-mRNA in splenocytes was quantified by flow cytometry. The data indicated that on average 85% of Cy5-mRNA is internalized by CD45+ splenocytes, which is equally distributed among dendritic cells, macrophages, and B cells (20%). Only a small proportion is localized to CD8+ T cells (5%) and CD4+ T cells (1%).

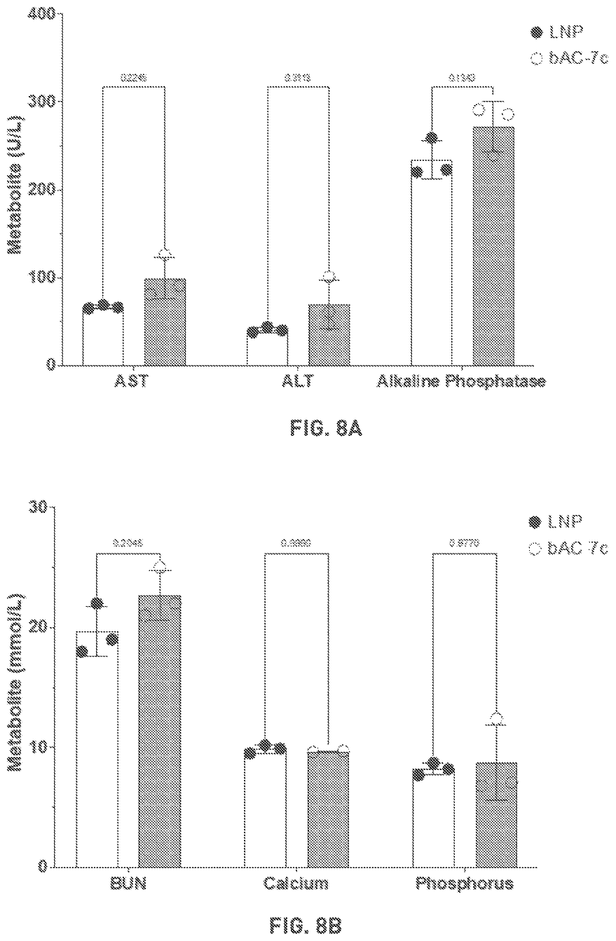

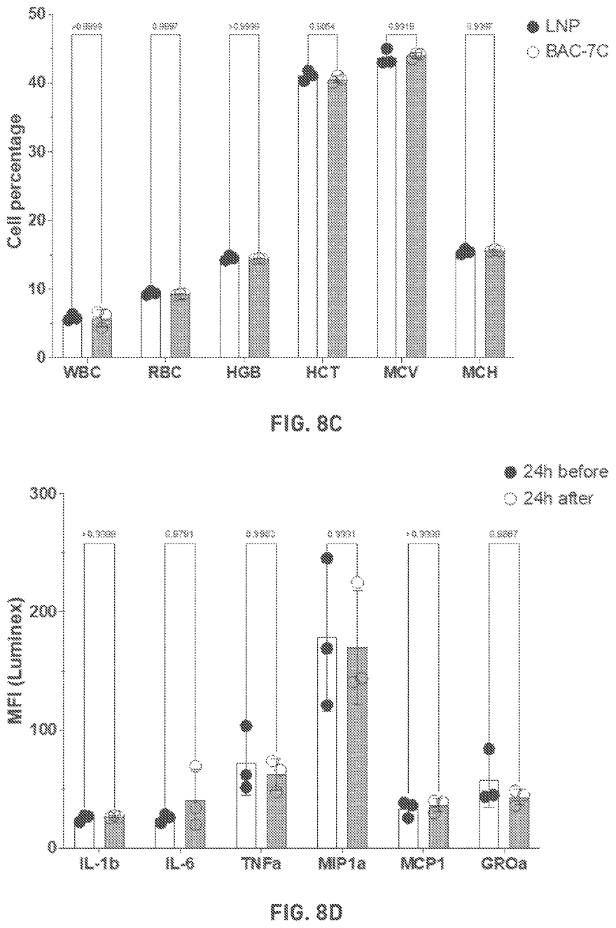

Assessing in vivo tolerability is a critical aspect in determining the clinical viability of drug delivery systems. Accordingly, a comprehensive pre-clinical toxicology blood analysis was conducted following the administration of bAC-7c/mRNA formulations. For comparison, FDA-approved MC3 LNP components were formulated with mRNA luciferase. When compared to MC3 formulations, bAC-7c formulations exhibited similar levels of blood metabolites indicative of liver damage, namely ALT, AST, and alkaline phosphatase (ALP), as shown in FIG. 8A.

Similarly, as shown in FIG. 8B, metabolites indicative of kidney damage, including blood urea nitrogen, calcium, and phosphorus, were also similar between the MC3 and bAC formulations. These findings suggest the absence of clinical signs of acute toxicity or alterations in clinical pathology.

A complete blood count (CBC) test was also performed on the mice following MC3 or bAC-7c mRNA delivery. Levels of red blood cells (RBC), white blood cells (WBC), hemoglobin (HGB), hematocrit (HCT), mean corpuscle volume (MCV), and mean corpuscle hemoglobin (MCH) were determined. FIG. 8C shows that the analytes of the CBC test for mice administered bAC-7c mRNA remained normal and were comparable to the MC3 formulation.

Similarly, as shown in FIG. 8D, the levels of inflammatory cytokines, including IL-1b, IL-6, and TNFa, remained similar 24 hours before and after bAC-7c mRNA delivery.

Unless otherwise stated, statistical analysis was performed with Prism (GraphPad Software v9.2.0). For comparing two groups, P values were determined using Student's t-tests (two-tailed). For comparing more than two groups, one-way ANOVAs followed by Tukey's test were applied. Differences between groups were considered significant for P values <0.05. No statistical methods were used to predetermine sample sizes. Mice were assigned to the various experimental groups randomly. Data collection and analysis were not performed blind to the conditions of the experiments.

In summary, the results described here demonstrate that bAC compounds are well-tolerated, exhibit spleen tropism without the need for targeting ligands, and can mediate efficient mRNA delivery to T lymphocytes. These data support the utility of the bAC compounds as promising vectors for in vivo delivery of nucleic acids, including mRNA, as well as delivery to splenic T cells.

### Definitions

Unless the context indicates otherwise, it is specifically intended that the various features of the disclosure described herein can be used in any combination. Moreover, the disclosure also contemplates that in some aspects any feature or combination of features set forth herein can be excluded or omitted. To illustrate, if the specification states that a complex has components A, B and C, it is specifically intended that any of A, B or C, or a combination thereof, can be omitted and disclaimed singularly or in any combination.

It must be noted that as used herein and in the appended claims, the singular forms “a”, “an”, and “the” include plural referents unless the context clearly dictates otherwise. Thus, for example, reference to “a cancer cell” includes a plurality of cancer cells. In other examples, reference to “a nucleic acid” or “nucleic acid” includes a plurality of nucleic acid molecules, i.e. nucleic acids.

The term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about means the specified value.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited aspect. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The abbreviations used herein have their conventional meaning within the chemical and biological arts. The chemical structures and formulae set forth herein are constructed according to the standard rules of chemical valency known in the chemical sciences.

The terms “oligomer” and “polymer” are used interchangeably herein to refer to a compound that has a plurality of repeating subunits, which may be referred to as blocks or monomer units, or simply as monomers. The terms “co-oligomer” or “copolymer” are used interchangeably herein to refer to an oligomer or polymer that includes two or more different types of monomers. For example, in the context of the present invention the compounds provided are co-oligomers, which may also be referred to as copolymers, comprising at least two different types of monomers, a lipid functionalized bAC monomer and an alpha aminoester monomer. Thus, the co-oligomers described herein may also be referred to herein as co-oligomers of cationic alpha aminoester and lipophilic bAC monomer repeating units.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “block copolymer” is used in accordance with its ordinary meaning and refers to two or more portions (e.g., blocks) of polymerized monomers linked by a covalent bond. In aspects, a block copolymer is a repeating pattern of polymers. In aspects, the block copolymer includes two or more monomers in a periodic (e.g., repeating pattern) sequence. For example, a diblock copolymer has the formula: -B-B-B-B-B-B-A-A-A-A-A-, where ‘B’ is a first subunit and ‘A’ is a second subunit covalently bound together. A triblock copolymer therefore is a copolymer with three distinct blocks, two of which may be the same (e.g., -A-A-A-A-A-B-B-B-B-B-B-A-A-A-A-A-) or all three are different (e.g., -A-A-A-A-A-B-B-B-B-B-B-C-C-C-C-C-) where ‘A’ is a first subunit, ‘B’ is a second subunit, and ‘C’ is a third subunit, covalently bound together. The term “random copolymer” refers to monomers randomly linked together in the polymer chain. The terms “random copolymer” and “statistical copolymer” are used interchangeably. The compounds described herein may be block or random copolymers. For example, a diblock lipid could be a blocked or random mixture of two lipids. Similarly, a triblock copolymer comprising two lipid blocks and a cationic block could be blocked or random with respect to the sequence of monomer residues.

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The terms “nucleic acid” and “polynucleotide” may be used interchangeably to refer to deoxyribonucleotides or ribonucleotides and polymers thereof in either single-, double- or multiple-stranded form, or complements thereof. The terms “polynucleotide,” “oligonucleotide,” and “oligo” are also used interchangeably. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides include single and double stranded DNA, single and double stranded RNA, and hybrid molecules having mixtures of single and double stranded DNA and RNA. RNA may include messenger RNA (mRNA), small interference RNA (siRNA), short hairpin RNA (shRNA), micro RNA (miRNA), guide RNA (gRNA), CRISPR RNA (crRNA), and transactivating RNA (tracrRNA). DNA may include plasmid DNA (pDNA), minicircle DNA, genomic DNA (gNDA), and fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids has one or more arms or branches of nucleotides. Optionally, the branched nucleic acids are repetitively branched to form higher ordered structures such as dendrimers and the like.

Polynucleotides may comprise known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Contacting” is used in accordance with its plain ordinary meaning and refers to the process of allowing at least two distinct species (e.g. chemical compounds including biomolecules or cells) to become sufficiently proximal to react, interact, associate, or physically touch. It should be appreciated, however, that the resulting reaction product can be produced directly from a reaction between the added reagents or from an intermediate from one or more of the added reagents which can be produced in the reaction mixture. In aspects, contacting includes, for example, allowing a nucleic acid to interact with an endonuclease.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, having the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “cationic charge altering releasable transporter (“CART”)” refers to the co-oligomer portion of the compositions disclosed herein. The CART compounds form stable nanoparticulate complexes with nucleic acid molecules, which may be referred to herein as nucleic acid “cargo” or simply “cargo”. Such nanoparticles are generally in a size range of from 50-400 nanometers (nm) or from about 100-300 nm. In aspects, a CART and its nucleic acid cargo form nanoparticles having a mean particle size (Z) in the range of 100-300 nanometers (nm), a distribution size of from 100-300 nm, a polydispersity index of from 0.10-0.040, and a Zeta potential of from about 10-50 mV, preferably from about 10-30 mV. CARTs advantageously deliver their cargo across cell membranes followed by rapid and efficient release via an intramolecular rearrangement of the poly(alpha-amino ester) component of the co-oligomer, which occurs very rapidly, e.g., in less than 5 minutes, at intracellular pH, e.g., pH of about 7.4. In aspects, the co-oligomer degrades rapidly at pH 7.4 with a half-life of less than 20 min, preferably less than 10 min or less than 5 min.

The term “amphipathic polymer” as used herein refers to a polymer containing both hydrophilic and hydrophobic portions. In aspects, the hydrophilic to hydrophobic portions are present in a 1 to 1 mass ratio. In aspects, the hydrophilic to hydrophobic portions are present in a 1 to 2 mass ratio. In aspects, the hydrophilic to hydrophobic portions are present in a 1 to 5 mass ratio. In aspects, the hydrophilic to hydrophobic portions are present in a 2 to 1 mass ratio. In aspects, the hydrophilic to hydrophobic portions are present in a 5 to 1 mass ratio. An amphipathic polymer may be a diblock or triblock copolymer. In aspects, the amphiphilic polymer may include two hydrophilic portions (e.g., blocks) and one hydrophobic portion (e.g., block)

The term “initiator” refers to a compound that is involved in a reaction synthesizing a co-oligomer having the purpose of initiating the polymerization reaction. Thus, the initiator is typically incorporated at the end of a synthesized polymer. For example, a plurality of molecules of one type (or formula) of monomer or more than one type of monomers (e.g. two different types of monomers) can be reacted with an initiator to provide a co-oligomer. The initiator can be present on at least one end of the resulting polymer and not constitute a repeating (or polymerized) unit(s) present in the polymer.

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The foregoing definitions are provided to facilitate understanding of certain terms used frequently herein.

1. A compound Formula (I): 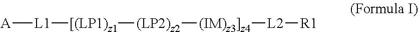
wherein:
A is a substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl;
or
A is a ligand moiety that binds to and is internalized by a cell surface receptor, optionally a saccharide, a disaccharide, an oligosaccharide, a liposaccharide, a lipid, a peptide, an antibody, or a small molecule;
R1 is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L1 and L2 are optional and each is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP1 and LP2 are each independently a functionalized beta-amido carbonate (bAC) monomer unit having a structure defined by 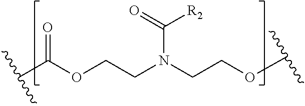
wherein
R2 is branched or unbranched, substituted or unsubstituted C₈-C₃₀ alkyl or heteroalkyl, which may be fully saturated, mono- or polyunsaturated;
IM is an alpha aminoester monomer unit having a structure defined by 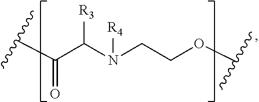
where R3 is dihydrogen or methyl and R4 is dihydrogen or substituted or unsubstituted C₂-C₅ heteroalkyl, wherein the heteroatom is nitrogen and wherein the substituent, if present, is carbonyl;
and
z1 and z2 are independently 0 to 100, optionally from 5-20, and at least one of z1 or z2 is not 0;
z3 is 2-100, optionally 5-20; and
z4 is 1-100.

2. The compound of claim 1, wherein IM is

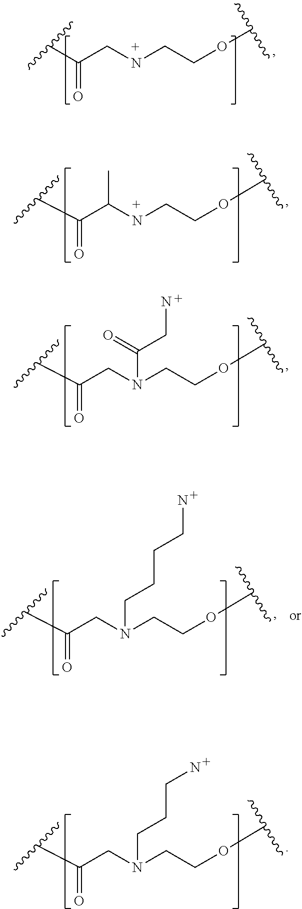

3. The compound of claim 1, wherein A is alkoxyaryl, optionally methoxyphenyl or ethoxyphenyl, and L1 is a bond.

4. The compound of claim 1, wherein the compound has a structure of Formula Ia: 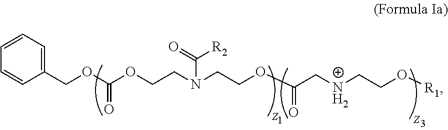
wherein R1 is hydrogen and R2 is branched or unbranched, substituted or unsubstituted C₈-C₃₀ alkyl or heteroalkyl, which may be fully saturated, mono- or polyunsaturated.

5. The compound of claim 4, wherein R² is unsubstituted branched or unbranched C₈-C₃₀ alkyl, which may be fully saturated, mono- or polyunsaturated.

6. The compound of claim 4, wherein R2 is substituted or unsubstituted branched or unbranched C₈-C₃₀ heteroalkyl and the heteroatom is O.

7. The compound of claim 4, wherein R2 is substituted branched or unbranched C₈-C₃₀ alkyl or heteroalkyl containing from 1 to 6 substituents, optionally selected from one or more of methyl and acyl.

8. The compound of claim 1, wherein A is a ligand moiety that binds to a cell surface receptor selected from a saccharide, a disaccharide, an oligosaccharide, a liposaccharide, a lipid, a peptide, an antibody, or a small molecule, optionally fingolimod or a fingolimod analog.

9. The compound of claim 8, wherein A is glucose (beta-D-glucopyranoside) or galactose (alpha-D-galactopyranose).

10. The compound of claim 8, wherein A is

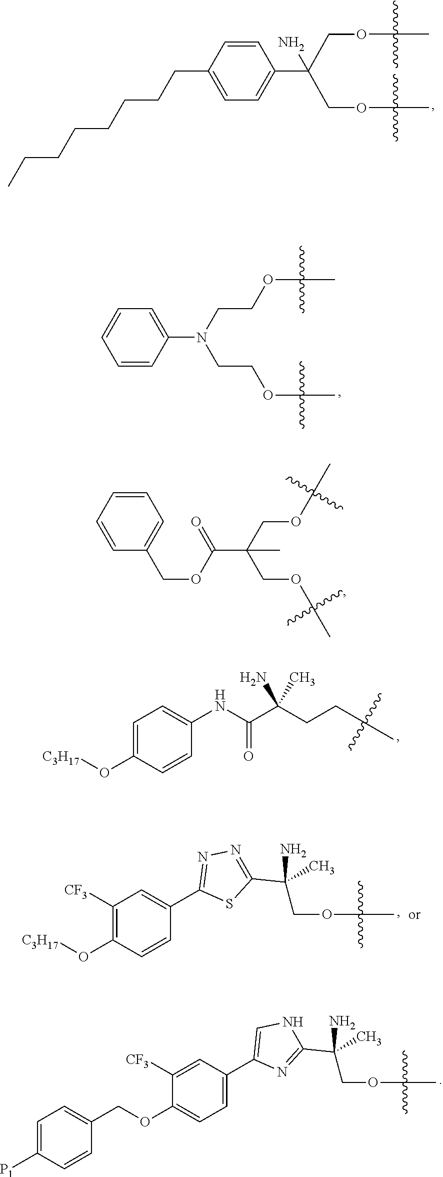

11. The compound of claim 1, wherein R2 is stearyl, oleyl, linoleyl, dodecyl, nonenyl, or isoprenyl.

12. The compound of claim 1, wherein R² is

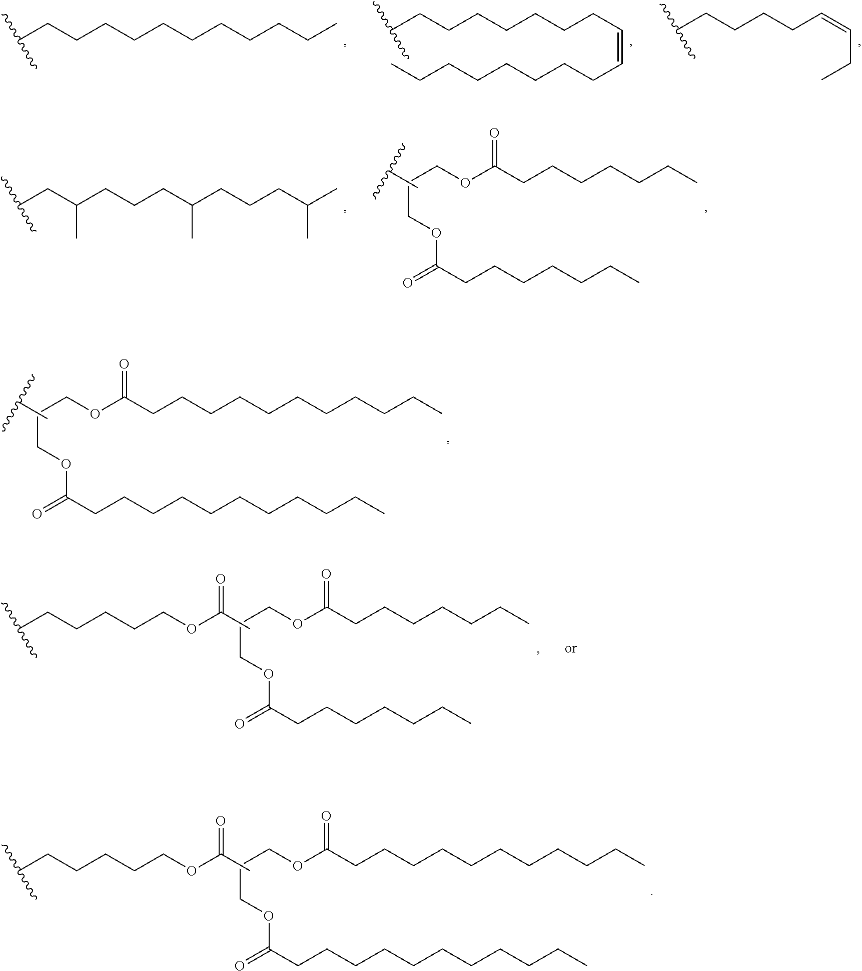

13. The compound of claim 4, wherein R2 is isoamyl, phytyl, farnesyl, or

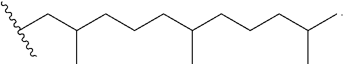 [SMILES: *CC(C)CCCC(C)CCCC(C)C]

14. The compound of claim 13, wherein z1 is 5-25 and z3 is 10-20; optionally wherein z1 is 17 and z3 is 10, which compound is designated bAC-4a; or wherein z1 is 10 and z3 is 11, which compound is designated bAC-4b; or wherein z1 is 9 and z3 is 16, which compound is designated bAC-4c.

15. The compound of claim 4, wherein R2 is

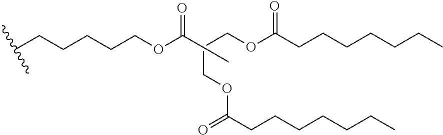

16. The compound of claim 15, wherein z1 is 5-25 and z3 is 10-20; optionally wherein z1 is 17 and z3 is 10, which compound is designated bAC-7a; or wherein z1 is 10 and z3 is 10, which compound is designated bAC-7b; or wherein z1 is 12 and z3 is 18, which compound is designated bAC-7c.

17. The compound of claim 4, wherein R2 is

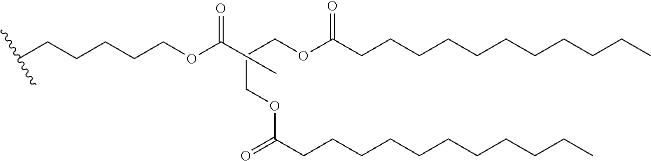

18. The compound of claim 17, wherein z1 is 5-25 and z3 is 5-20; optionally wherein z1 is 16 and z3 is 9, which compound is designated bAC-8a; or wherein z1 is 10 and z3 is 9, which compound is designated bAC-8b; or wherein z1 is 8 and z3 is 18, which compound is designated bAC-8c.

19. A composition comprising a compound of claim 1, non-covalently attached to a nucleic acid.

20. The composition of claim 19, wherein the nucleic acid is RNA or DNA.

21. The composition of claim 19, wherein the nucleic acid is selected from the group consisting of messenger RNA (mRNA), small interference RNA (siRNA), short hairpin RNA (shRNA), micro RNA (miRNA), guide RNA (gRNA), CRISPR RNA (crRNA), transactivating RNA (tracrRNA), circular RNA (circRNA), self-amplifying RNA, plasmid DNA (pDNA), minicircle DNA, and genomic DNA (gNDA), and combinations of two or more of any of the foregoing.

22. The composition of claim 19, wherein the nucleic acid is messenger RNA (mRNA).

23. The composition of claim 22, wherein the mRNA encodes a chimeric antigen receptor (CAR).

24. A pharmaceutical composition comprising the composition of claim 19, and a pharmaceutically acceptable carrier or excipient.

25. The pharmaceutical composition of claim 24, wherein the composition is a vaccine composition, optionally, wherein the carrier or excipient comprises an immunological adjuvant.

26. A method for transfecting a nucleic acid into a cell, the method comprising contacting the cell with the composition of claim 19, wherein the contacting is in vivo, in vitro, or ex vivo.

27. The method of claim 26, wherein the cell is a eukaryotic cell, a mammalian cell, a tumor cell, or a lymphoid cell.

28. The method of claim 26, wherein the cell is a B lymphocyte, a dendritic cell, a macrophage cell, or a T lymphocyte, optionally wherein the T lymphocyte is a CD8+ T cell or a CD4+ T cell.

29. A method for transfecting a nucleic acid into a splenocyte cell of a mammal, the method comprising administering to the mammal the composition of any one of claims 19 to 23, or the pharmaceutical composition of claim 24, optionally wherein the nucleic acid is an mRNA.

30. A method for treating an autoimmune disease or disorder, a cancer, or an infectious disease, the method comprising administering the pharmaceutical composition of claim 24 to a subject in need of therapy for an autoimmune disease or disorder, a cancer, or an infectious disease, wherein the nucleic acid is a therapeutic nucleic acid or encodes a therapeutic protein, optionally wherein administration is by a parenteral route, further optionally wherein administration is by an intravenous route, optionally wherein the subject is human.

31. A method for preventing a disease or disorder, the method comprising administering the pharmaceutical composition of claim 25 to a subject in need of therapy for the disease or disorder, wherein the nucleic acid encodes one or more antigenic or immunogenic epitopes or peptides, optionally wherein administration is by a parenteral route, further optionally wherein administration is by an intravenous route, optionally wherein the subject is human.

32. The method of claim 31, wherein the disease or disorder is an infectious disease.

33. The method of claim 32, wherein the infectious disease is caused by an infectious agent, optionally wherein the infectious agent is a virus, a bacterium, or a protozoa.

34-36. (canceled)
